SEQUENCE LISTING (4 sequences)

SEQ ID NO: 1 - protein, 107 aa, Artificial Sequence
  Light chain variable region
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 2 - protein, 122 aa, Artificial Sequence
  Heavy chain variable region
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 3 - protein, 214 aa, Artificial Sequence
  Light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 452 aa, Artificial Sequence
  Heavy chain
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2023/0338526 A1
(43) Pub. Date: Oct. 26, 2023

(54) ANTI-CD20 ANTIBODY FORMULATION AND USE OF ANTI-CD20 ANTIBODY FOR TREATMENT OF CD20 POSITIVE DISEASES

(71) Applicant: Bio-Thera Solutions, Ltd., (US)

(72) Inventors: Yanyu CHEN, Guangzhou, Guangdong (CN); Yong WU, Guangzhou, Guangdong (CN); Yuanqing ZHOU, Guangzhou, Guangdong (CN); Chao QIN, Guangzhou, Guangdong (CN); Cuizhen XIAO, Guangzhou, Guangdong (CN); Zhihao WU, Guangzhou, Guangdong (CN); Dandan HUANG, Guangzhou, Guangdong (CN); Yujie LIU, Guangzhou, Guangdong (CN); Shengwu WANG, Guangzhou, Guangdong (CN); Cuihua LIU, Guangzhou, Guangdong (CN); Yunpeng QI, Guangzhou, Guangdong (CN); Jin-Chen YU, Guangzhou, Guangdong (CN); Li ZHANG, Guangzhou, Guangdong (CN); Shengfeng LI, Guangzhou, Guangdong (CN)

(21) Appl. No.: 17/767,990

(22) PCT Filed: Oct. 12, 2020
(86) PCT No.: PCT/CN2020/120461

§ 371 Date: Apr. 11, 2022

(30) Foreign Application Priority Data
Oct. 12, 2019 (WO) PCT/CN2019/110843
Oct. 12, 2019 (WO) PCT/CN2019/110847
Oct. 18, 2019 (CN) 201910991541.8

(51) Int. Cl. A61K 39/395; A61K 9/08; C07K 16/28; A61K 47/26; A61K 47/22; A61P 37/06

(52) U.S. Cl. CPC A61K 39/39591; A61K 9/08; C07K 16/2887; A61K 47/26; A61K 47/22; A61P 37/06; A61K 2039/505

(57) ABSTRACT

Disclosed are anti-CD20 antibody formulations and use of anti-CD20 antibodies for the treatment of CD20 positive diseases, such as methods of using anti-CD20 antibodies for treating CD20 positive diseases, such as neuromyelitis optica spectrum disorders (NMOSD), non-Hodgkin's lymphoma (NHL), multiple sclerosis (MS), immune thrombocytopenia (ITP), rheumatoid arthritis (RA), Wegener's granulomatosis (WG), microscopic polyangiitis (MPA), lupus nephritis, systemic lupus erythematosus and chronic lymphocytic leukemia (CLL).

### REFERENCE TO SEQUENCE LISTING

The present application is being filed along with a Sequence Listing in electronic format. The Sequence Listing is provided as a file entitled DSP1P202582J_SequenceListing, created on Sep. 17, 2020, which is 8 kilobytes in size. The information in the electronic format of the Sequence Listing is incorporated herein by reference in its entirety.

### FIELD

The present invention belongs to the field of biopharmaceutical and relates to anti-CD20 antibody formulations and use of anti-CD20 antibodies for the treatment of CD20 positive diseases, such as methods of using anti-CD20 antibodies for treating CD20 positive diseases, such as neuromyelitis optica spectrum disorders (NMOSD), non-Hodgkin's lymphoma (NHL), multiple sclerosis (MS), immune thrombocytopenia (ITP), rheumatoid arthritis (RA), Wegener's granulomatosis (WG), microscopic polyangiitis (MPA), lupus nephritis, systemic lupus erythematosus and chronic lymphocytic leukemia (CLL).

### BACKGROUND

CD20 molecule is a non-glycosylated phosphoprotein specifically labeled on the surface of human lymphocyte subgroup (B cell group). It consists of 297 amino acids with a molecular weight of 33-37 kD, and is expressed on the surface of more than 95% of B cells. CD20 molecule exists in both normal B cells and malignant cells, and is especially expressed in more than 90% of B-cell non-Hodgkin's lymphoma. The CD20 molecule has four transmembrane regions, and the amino terminus and the carboxy terminus are located on the inner side of the plasma membrane. Between the third transmembrane region and the fourth transmembrane region, there is a loop region composed of 43 amino acid residues, which constitutes the main epitope. The CD20 antigen molecule is relatively exposed and accessible. When CD20 is close to each other under the action of antibodies, the polymer formed by cross-linking or even super-crosslinking functions as a calcium ion channel, allowing extracellular calcium ions to flow into the cells: in addition, the tyrosine protein kinases of the Src family activate each other due to proximity. Signaling pathways are initiated and endogenous calcium stores are mobilized, both of which lead to an increase in intracellular calcium ion concentration and then affect the operation of cell cycle, regulate cell proliferation and differentiation and even lead to the occurrence of apoptosis.

Although the actual role of CD20 in promoting the proliferation and differentiation of B cells is not clear, CD20 provides an important target for antibody-mediated therapy, which can be used for controlling the B cells involved in cancers and autoimmune diseases.

Compared with traditional organic and inorganic drugs, an anti-CD20 antibody is larger and more complicated. In order to maintain the biological activity of the antibody, a formulation needs to maintain the integrity of the whole conformation of the amino acid core sequence of the protein, and prevent the degradation of multiple functional groups of the antibody at the same time.

Neuromyelitis optica (NMO) is an immune-mediated central nervous system (CNS) inflammatory demyelinating disease mainly involving the optic nerve and spinal cord, mostly characterized by severe optic neuritis (ON) and longitudinally extensive transverse myelitis (LETM). With in-depth research, the clinical features of NMO also include some non-optical and non-spinal performance. The etiology of NMO is primarily associated with aquaporin 4 antibody (AQP4-IgG). In addition, there is a group of limited forms of demyelinating diseases that do not meet the NMO diagnostic criteria in clinic. They have similar pathogenesis and clinical features as NMO, and some cases eventually evolved into NMO. In 2007, the above diseases were collectively named neuromyelitis optica spectrum disorders (NMOSD). In 2015, the International Panel for NMO Diagnosis (IPND) developed new international consensus on the diagnostic criteria for the NMOSD, and unified the terms NMO and NMOSD to be NMOSD. Wingerchuk, et al., Neurology; 2015, 85(2): 177-189.

### SUMMARY

On this basis, the present invention provides anti-CD20 antibody formulations and use of anti-CD20 antibodies for the treatment of CD20 positive diseases.

In the first aspect, the present invention provides a liquid formulation comprising an anti-CD20 antibody, which comprises the following ingredients: 10 mg/ml to 120 mg/ml anti-CD20 antibody, a buffer, a stabilizer and a surfactant, and the liquid formulation has a pH value ranging from 5 to 7.

In some embodiments, the solvent of the antibody formulation is water. In some embodiments, the solvent of the antibody formulation is sterile water for injection.

In some embodiments, the liquid formulation comprises 10 mg/ml to 120 mg/ml anti-CD20 antibody, 10 mM to 30 mM buffer, 58 mM to 292 mM stabilizer and 0.1 mg/ml to 0.5 mg/ml surfactant, and the liquid formulation has a pH value ranging from 5.5 to 6.5.

In some embodiments, the liquid formulation comprises 15 mg/ml to 80 mg/ml anti-CD20 antibody, 10 mM to 30 mM buffer, 80 mM to 240 mM stabilizer and 0.1 mg/ml to 0.4 mg/ml surfactant, and the liquid formulation has a pH value ranging from 5.5 to 6.2.

In some embodiments, the buffer is selected from succinate buffer, citrate buffer, phosphate buffer, histidine buffer and acetate buffer.

In some embodiments, the stabilizer is selected from sucrose, trehalose, sorbitol, mannitol and methionine; and the surfactant is selected from polysorbate-80 and polysorbate-20.

In some embodiments, the succinate buffer comprises succinic acid and sodium succinate, the citrate buffer comprises citric acid and sodium citrate, the histidine buffer comprises L-histidine and L-histidine hydrochloride and the acetate buffer comprises acetic acid and sodium acetate.

In some embodiments, the concentration of the anti-CD20 antibody is about 15 mg/ml, 40 mg/ml, 60 mg/ml, 80 mg/ml (i.e., 8%), or a number or a range between any two of these values (end point values included); the concentration of the buffer is about 10 mM, 15 mM, 26 mM, 30 mM, or a number or a range between any two of these values (end point values included); the concentration of the stabilizer is about 80 mM, 140 mM, 190 mM, 240 mM, or a number or a range between any two of these values (end point values included); the concentration of the surfactant is about 0.1 mg/nil, 0.3 mg/ml, 0.4 mg/ml (i.e., 0.04%), or a number or a range between any two of these values (end point values included); and the pH value is about 5.5, 5.6, 5.7, 6.0, 6.2, or a number or a range between any two of these values (end point values included).

In some embodiments, the anti-CD20 antibody is selected from a monoclonal antibody and a CD20-binding fragment.

In some embodiments, the anti-CD20 antibody comprises:
a light chain variable region (VL) comprising the amino acid sequence of SEQ ID NO: 1 or a polypeptide having at least 80% sequence identity to SEQ ID NO: 1; and
a heavy chain variant region (VH) comprising an amino acid sequence of SEQ ID NO: 2 or a polypeptide having at least 80% sequence identity to SEQ ID NO: 2.

In some embodiments, the VL comprises a polypeptide having at least 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values (end points included), sequence identity to SEQ ID NO: 1. In some embodiments, the VH comprises a polypeptide having at least 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values (end points included), sequence identity to SEQ ID NO:2.

In some embodiments, the VL comprises the amino acid sequence of SEQ ID NO: 1, and the VH comprises the amino acid sequence of SEQ ID NO: 2.

In some embodiments, the anti-CD20 antibody comprises:
a light chain comprising the amino acid sequence of SEQ ID NO: 3 or a polypeptide having at least 80% sequence identity to SEQ ID NO: 3; and
a heavy chain comprising the amino acid sequence of SEQ ID NO: 4 or a polypeptide having at least 80% sequence identity to SEQ ID NO: 4.

In some embodiments, the light chain comprises a polypeptide having at least 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values (end points included), sequence identity to SEQ ID NO: 3. In some embodiments, the heavy chain comprises a polypeptide having at least 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values (end points included), sequence identity to SEQ ID NO:4.

In some embodiments, the anti-CD20 antibody comprises:
a light chain comprising the amino acid sequence of SEQ ID NO: 3; and
a heavy chain comprising the amino acid sequence of SEQ ID NO: 4.

In some embodiments, the anti-CD20 antibody comprises:
two light chains, each comprising the amino acid sequence of SEQ ID NO: 3; and
two heavy chains, each comprising the amino acid sequence of SEQ ID NO: 4.

light chain variable region (SEQ ID NO: 1)
EIVLTQSPATLSLSPGERATLSCRASQSVSSYLAWYQQKPGQAPRLLIY
DASNRATGIPARFSGSGSGTDFTLTISSLEPEDFAVYYCQQRSNWPITF
GQGTRLEIK
heavy chain variant region (SEQ ID NO: 2)
EVOLVESGGGLVQPGRSLRLSCAASGFTFNDYAMHWVRQAPGKGLEWVS
TISWNSGSIGYADSVKGRFTISRDNAKKSLYLQMNSLRAEDTALYYCAK
DIQYGNYYYGMDVWGQGTTVTVSS
light chain (SEQ ID NO: 3)
EIVLTQSPATLSLSPGERATLSCRASQSVSSYLAWYQQKPGQAPRLLIY
DASNRATGIPARFSGSGSGTDFTLTISSLEPEDFAVYYCQQRSNWPITF
GQGTRLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKS FNRGEC
heavy chain (SEQ ID NO: 4)
ADSVKGRFTISRDNAKKSLYLQMNSLRAEDTALYYCAKDIQYGNYYYGM
DVWGQGTTVTVSSASTKGPSVFPLAPGSSKSTSGTAALGCLVKDYFPEP
VTVSWNSGALTSGVHTFPAVLQSSGLYSLSSVVTVPSSSLGTQTYICNV
NHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK

In some embodiments, the anti-CD20 antibody is BAT4406F which comprises:
a light chain comprising the amino acid sequence of SEQ ID NO: 3; and
a heavy chain comprising the amino acid sequence of SEQ ID NO: 4;
and is characterized with a very low (no more than 5%) fucose content.

In some embodiments, the anti-CD20 antibody is BAT4406F which comprises:
two light chains, each comprising the amino acid sequence of SEQ ID NO: 3; and two heavy chains, each comprising the amino acid sequence of SEQ ID NO: 4;
and is characterized with a very low (no more than 5%) fucose content.

BAT4406F is a glycosylation-optimized IgG₁ subclass of fully human monoclonal antibody described in International Patent Application PCT/CN2018/100008.

In some embodiments, the anti-CD20 mAb or the antigen-binding fragment thereof (e.g., BAT4406F) is produced from a cell line having α-(1,6)-fucosyltransferase gene knocked out, which can express antibodies having a low fucose content (e.g., 0-5%) and an enhanced ADCC effect. In some embodiments, the cell line is CHO-BAT-KF cell line described in International Patent Application PCT/CN2018/100008. The taxonomic denomination of the CHO host cell strain is Chinese hamster overy cells CHO-BAT-KF FUT8(−/−). An anti-CD20 mAb (e.g., BAT4406F) or an antigen binding fragment thereof can be expressed and purified as described in International Patent Application PCT/CN2018/100008, Purification can be carried out by conventional methods, such as centrifuging the cell suspension and harvesting the supernatant, which can be further cleared by centrifuging. Protein A affinity and ion exchange columns can be used to purify the antibodies.

In some embodiments, the glycosylation of the anti-CD20 mAb (e.g., BAT4406F) is characterized by one or more of the following:
the fucose content of the antibody is very low (e.g., 0-5%);
the galactose level of the antibody is low (e.g., ≤30%);
the mannose level of the antibody is low (e.g., ≤5%);
the high mannose level of the antibody is low (e.g., ≤5%);
the G0 level of the antibody is high (e.g., ≥60%).

In some embodiments, the antibody has a low galactose level, e.g., ≤5%.

In some embodiments, the antibody has a high G0 level, e.g., ≥80%.

In some embodiments, the glycosylation site of the antibody is an Asn residue on the heavy chain, such as Asn297 in SEQ ID NO: 4.

In some embodiments, at least about 50% of the anti-CD20 mAb (e.g., BAT4406F) can comprise the G0 glycan at the asparagine residue 297 (Asn297) of SEQ ID NO: 4, and/or at most about 10% of the anti-CD20 mAb can comprise the Man5 glycan at the amino acid residue 297 of SEQ ID NO: 4. In some embodiments, less than about 20% of the anti-CD20 mAb comprise a fucosyl residue. In some embodiments, less than about 10% of the anti-CD20 mAb comprise a fucosyl residue. In some embodiments, less than about 5% of the anti-CD20 mAb comprise a fucosyl residue. In some embodiments, less than about 1% of the anti-CD20 mAb comprise a fucosyl residue.

In some embodiments, at least 50% of the anti-CD20 mAb (e.g., BAT4406F) comprises the G0 glycan at an N-glycosylation site of a constant region of the heavy chain of the anti-CD20 antibody. In some embodiments, at least 60% of the anti-CD20 mAb comprises the G0 glycan at an N-glycosylation site of a constant region of the heavy chain of the anti-CD20 antibody. In some embodiments, at least about 65% of the anti-CD20 mAb can comprise the G0 glycan at the N-glycosylation site of the constant region of the heavy chain of the anti-CD20 antibody. In some embodiments, at most 7% of the anti-CD20 mAb can comprise the Man5 glycan at the N-glycosylation site of the constant region of the heavy chain of the anti-CD20 antibody. In some embodiments, at most about 5% of the anti-CD20 mAb can comprise the Man5 glycan at the N-glycosylation site of the constant region of the heavy chain of anti-CD20 antibody. In some embodiments, at most about 3% of the anti-CD20 mAb can comprise the Man5 glycan at the N-glycosylation site of the constant region of the heavy chain of anti-CD20 antibody.

In some embodiments, the oligosaccharides of the Fc region of the anti-CD20 mAb (e.g., BAT4406F) in the formulation comprise no more than about 10% of fucosyl content. In some embodiments, the oligosaccharides of the Fc region of the anti-CD20 mAb comprise no more than about 5% of fucosyl content. In some embodiments, the oligosaccharides of the Fe region of the anti-CD20 mAb comprise no more than about 1% of fucosyl content.

In some embodiments, at least, or at least about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, or a number or a range between any two of these values (end points included), of the anti-CD20 mAb in the formulation can comprise the G0 glycan at the N-glycosylation site of the constant region of the heavy chain of the anti-CD20 mAb. In some embodiments, at most, or at most about, 1%, 2%, 3%, 4%, 5%, 6%, 7%, 8%, 9%, 10%, 11%, 12%, 13%, 14%, 15%, 16%, 17%, 18%, 19%, 20%, or a number or a range between any two of these values (end points included), of the anti-CD20 mAb in the formulation can comprise any glycans other than the G0 glycan at the N-glycosylation site of the constant region of the heavy chain of the anti-CD20 mAb.

In some embodiments, at most, or at most about, 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%. 18%, 19%, 20%, or a number or a range between any two of these values, of the anti-CD20 mAb (e.g., BAT4406F) can comprise the G0-GN glycan at the N-glycosylation site of the constant region of the heavy chain of the anti-CD20 mAb. In some embodiments, at most, or at most about, 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, or a number or a range between any two of these values (end points included), of the anti-CD20 mAb can comprise the Man5 glycan at the N-glycosylation site of the constant region of the heavy chain of the anti-CD20 mAb. In some embodiments, at most, or at most about, 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, or a number or a range between any two of these values (end points included), of the anti-CD20 mAb can comprise the G1 glycan at the N-glycosylation site of the constant region of the heavy chain of the anti-CD20 mAb. In some embodiments, at most, or at most about, 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, or a number or a range between any two of these values (end points included), of the anti-CD20 mAb can comprise the G1′ glycan at the N-glycosylation site of the constant region of the heavy chain of the anti-CD20 mAb. In some embodiments, at most, or at most about, 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, or a number or a range between any two of these values (end points included), of the anti-CD20 mAb can comprise the G2 glycan at the N-glycosylation site of the constant region of the heavy chain of the anti-CD20 mAb.

In some embodiments, the fucose content of the anti-CD20 mAb (e.g., BAT4406F) in the formulation is very low. For example, the fucose content of the anti-CD20 mAb in the formulation is, is about, or is at most, 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, or a number or a range between any two of these values. In some embodiments, die anti-CD20 mAb (e.g., BAT4406F) in the formulation do not have any fucose residue, i.e., the formulation comprises nonfucosylated or afucosylated anti-CD20 mAb (e.g., nonfucosylated or afucosylated BAT4406F).

In some embodiments, the galactose level of the anti-CD20 mAb is low. For example, the galactose level of the antibody is, is about, or is at most, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, or a number or a range between any two of these values (end points included).

In some embodiments, the mannose content of the anti-CD20 mAb is very low. For example, the mannose content of the anti-CD20 mAb is, is about, or is at most, 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, or a number or a range between any two of these values (end points included).

In some embodiments, the high mannose content of the anti-CD20 mAb is very low. For example, the mannose content of the anti-CD20 mAb is, is about, or is at most, 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, or a number or a range between any two of these values (end points included).

In some embodiments, the G0 level of the anti-CD20 mAb is high. For example, the G0 level of the anti-CD20 mAb is, is about, or is at least, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%. 86%, 87%, 88%, 89%, 90%, 91%, 92%, 93%, 94%, 95%, 96%, 97%, 98%, 99%, 99.9%. or a number or a range between any two of these values (end points included).

In some embodiments, the liquid formulation comprises 20 mg/ml to 50 mg/ml anti-CD20 antibody; the concentration of the anti-CD20 antibody is about 20 mg/ml, 30 mg/ml, 40 mg/ml, 50 mg/ML or a number or a range between any two of these values (end points included).

In some embodiments, the liquid formulation comprises 18 mM to 22 mM histidine buffer; the concentration of the histidine buffer is about 18 mM, 19 mM, 20 mM, 22 mM, or a number or a range between any two of these values (end points included).

In some embodiments, the molar concentration ratio of the L-histidine to the L-histidine hydrochloride in the histidine buffer is 1 to 2:2.

In some embodiments, the liquid formulation comprises 158 mM to 225 mM trehalose; the concentration of the trehalose is about 158 mM, 158.6 mM, 170 mM, 220 mM, 224.6 mM, 225 mM (224.6 mM is 7.68%), or a number or a range between any two of these values (end points included).

In some embodiments, the liquid formulation comprises 0.18 mg/ml to 0.22 mg/ml polysorbate-80; the concentration of the polysorbate-80 is about 0.18 mg/ml, 0.19 mg/ml, 0.2 mg/ml, 0.22 mg/ml (i.e., 0.022%), or a number or a range between any two of these values (end point values included).

In some embodiments, the pH value of the liquid formulation ranges from 5.7 to 5.9; and the pH value is about 5.7, 5.8, 5.9, or a number or a range between any two of these values (end point values included).

In some embodiments, the liquid formulation comprises the following ingredients: about 20 mg/ml anti-CD20 antibody, about 20 mM histidine buffer, about 158.6 mM trehalose (i.e., 54 mg/ml trehalose) and about 0.2 mg/ml polysorbate-80, and the pH value of the liquid formulation is about 5.8; and based on the pH value of 5.8, the molar ratio of the L-histidine to the L-histidine hydrochloride in the histidine buffer is 2:3.

In some embodiments, the liquid formulation comprises the following ingredients: about 20 mg/ml anti-CD20 antibody, about 20 mM histidine buffer, about 224.6 mM trehalose (i.e., 76.8 mg/ml trehalose) and about 0.2 mg/ml polysorbate-80, and the pH value of the liquid formulation is about 5.8; and based on the pH value of 5.8, the molar ratio of the L-histidine to the L-histidine hydrochloride in the histidine buffer is 2:3.

In some embodiments, the liquid formulation comprises the following ingredients: about 50 mg/ml anti-CD20 antibody, about 20 mM histidine buffer, about 224.6 mM trehalose (i.e., 76.8 mg/ml trehalose) and about 0.2 mg/ml polysorbate-80, and the pH value of the liquid formulation is about 5.8; and based on the pH value of 5.8, the molar ratio of the L-histidine to the L-histidine hydrochloride in the histidine buffer is 2:3.

In some embodiments, the liquid formulation is a liquid formulation prepared according to Formulation 1, Formulation 2, Formulation 3 or Formulation 4.

In some embodiments, the liquid formulation can be used for injection, such as intravenous or subcutaneous injection.

In some embodiments, the liquid formulation prepared according to Formulation 1 is a formulation for intravenous injection.

In some embodiments, the liquid formulation prepared according to Formulation 2, 3 or 4 is a formulation for subcutaneous injection.

In some embodiments, the present invention further provides a formulation for intravenous injection, which comprises the above liquid formulation and a diluent (e.g., water (such as sterile water for injection), an isotonic solution (such as 0.9% NaCl solution for injection), etc.). In some embodiments, the ratio of the above liquid formulation to the diluent is 1:10 to 1:50, 1:20 to 1:40, or 1:25 to 1:35.

In the second aspect, the present invention provides a method for treating a disease involving cells expressing CD20 in a patient, comprises administering to a patient in need thereof an effective amount of the above liquid formulation.

In some embodiments, the patient is human.

In some embodiments, the patient is administrated by injection, such as intravenous or subcutaneous injection.

In some embodiments, when the patient is administrated by injection, the liquid formulation comprises the following ingredients: about 20 mg/ml anti-CD20 antibody, about 20 mM histidine buffer, about 158.6 mM trehalose and about 0.2 mg/ml polysorbate-80 and the pH value of the liquid formulation is about 5.8.

In some embodiments, when the patient is administrated by injection, the liquid formulation comprises the following ingredients: about 20 mg/ml anti-CD20 antibody, about 20 mM histidine buffer, about 224.6 mM trehalose and about 0.2 mg/ml polysorbate-80, and the pH value of the liquid formulation is about 5.8.

In some embodiments, when the patient is administrated by injection, the liquid formulation comprises the following ingredients: about 50 mg/ml anti-CD20 antibody, about 20 mM histidine buffer, about 224.6 mM trehalose and about 0.2 mg/ml polysorbate-80, and the pH value of the liquid formulation is about 5.8.

In some embodiments, the formulation is administered by intravenous injection, and the liquid formulation is diluted with a diluent (e.g., water (such as sterile water for injection), an isotonic solution (such as 0.9% NaCl solution for injection), etc.) before intravenous injection to the patient. In some embodiments, the above liquid formulation is diluted with the diluent in a ratio of 1:10 to 1:50, 1:20 to 1:40, or 1:25 to 1:35.

In some embodiments, the disease involving cells expressing CD20 is selected from a tumorigenic disease and an immune disease.

In some embodiments, the tumorigenic disease comprises B-cell lymphoma, the B-cell lymphoma comprises precursor B-cell lymphocytic leukemia/lymphoma and mature B-cell tumor, lymphoplasmacytic lymphoma, mantle cell lymphoma (MCL), low-grade, moderate-grade and high-grade follicular lymphomas, cutaneous follicle center lymphoma, MALT-type, nodular-type and splenic-type marginal zone B-cell lymphomas, hairy cell leukemia, diffuse large B-cell lymphoma, Burkitt lymphoma, plasmacytoma, plasma-cell myeloma, post-transplant lymphoproliferative disorders, Waldenstrom's macroglobulinemia and anaplastic large cell lymphoma (ALCL).

In some embodiments, the immune disease comprises psoriasis, psoriatic arthritis, dermatitis, systemic scleroderma and sclerosis, inflammatory bowel disease (IBD), Crohn's disease, ulcerative colitis, respiratory distress syndrome, meningitis, encephalitis, uveitis, glomerulonephritis, eczema, asthma, atherosclerosis, leukocyte adhesion deficiency, multiple sclerosis, Raynaud's syndrome, Sjogren's syndrome, juvenile-onset diabetes, Reiter's disease, Behcet's disease, immune complex nephritis, IgA nephropathy, IgM polyneuropathy, neuromyelitis optica, immune-mediated thrombocytopenia, hemolytic anemia, myasthenia gravis, lupus nephritis, systemic lupus erythematosus, rheumatoid arthritis (RA), atopic dermatitis, pemphigus, Graves' disease, Hashimoto's thyroiditis, Wegener's granulomatosis, Omenn's syndrome, chronic renal failure, acute infectious mononucleosis, and chronic obstructive pulmonary disease.

In some embodiments, the disease involving cells expressing CD20 is a disease or disorder treatable by B-cell depletion.

In some embodiments, the disease or disorder treatable by B-cell depletion is selected from the group consisting of cancers, anaphylaxis, cardiovascular diseases, inflammatory diseases, autoimmune diseases, metabolic diseases, neurological diseases, viral infections, and bacterial infections. For example, the disease may be cancer or anaphylaxis.

In some embodiments, the disease is selected from pernicious amenia (Addison's disease), amyotrophic lateral sclerosis, ankylosing spondylitis, erythema multiforme, lupus nephritis, dermatomyositis, immune-mediated thrombocytopenias, such as acute idiopathic thrombocytopenic purpurea and chronic idiopathic thrombocytopenic purpurea, Sydenham's chorea, rheumatic fever, polyglandular syndromes, Henoch-Schonlein purpura, post-streptococcal nephritis, erythema nodosum, Takayasu's arteritis, polyarteritis nodosa, Goodpasture's syndrome, thromboangitis ubiterans, primary biliary cirrhosis, Hashimoto's thyroiditis, thyrotoxicosis, chronic active hepatitis, polymyositis/dermatomyositis, polychondritis, pamphigus vulgaris, Wegener's granulomatosis, membranous nephropathy, tabes dorsalis, polymyaglia, pernicious anemia, rapidly progressive glomerulonephritis and fibrosing alveolitis, inflammatory diseases such as inflammatory skin diseases including psoriasis and dermatitis (e.g. atopic dermatitis); systemic scleroderma and sclerosis; diseases associated with inflammatory bowel disease (such as Crohn's disease and ulcerative colitis); respiratory distress syndrome (including adult respiratory distress syndrome; ARDS), dermatitis, meningitis, encephalitis, colitis, glomerulonephritis, uveitis, allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE); diabetes mellitus (e.g. Type 1 diabetes mellitus or insulin dependent diabetes mellitus); multiple sclerosis; Reynaud's syndrome; autoimmune thyroiditis; allergic encephalomyelitis; Sjorgen's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia etc.

In some embodiments, the disease is neuromyelitis optica spectrum disorders (NMOSD), non-Hodgkin's lymphoma (NHL), multiple sclerosis (MS), immune thrombocytopenia (ITP), rheumatoid arthritis (RA), Wegener's granulomatosis (WG), microscopic polyangiitis (MPA), lupus nephritis, systemic lupus erythematosus or chronic lymphocytic leukemia (CLL).

In some embodiments, the disease is refractory to or relapsed after at least one prior therapeutic regimen. In some embodiments, the disease is refractory to or relapsed after at least two prior therapeutic regimens.

In the third aspect, the present invention further provides use of the above liquid formulation for the preparation of a medicament for treating a disease involving cells expressing CD20 in a patient, wherein the disease involving cells expressing CD20 is selected from tumorigenic disease and immune disease.

In some embodiments, the patient is human.

In some embodiments, the tumorigenic disease comprises B-cell lymphoma, the B-cell lymphoma comprises precursor B-cell lymphocytic leukemia/lymphoma and mature B-cell tumor, lymphoplasmacytic lymphoma, mantle cell lymphoma (MCL), low-grade, moderate-grade and high-grade follicular lymphomas, cutaneous follicle center lymphoma, MALT-type, nodular-type and splenic-type marginal zone B-cell lymphomas, hairy cell leukemia, diffuse large B-cell lymphoma, Burkitt lymphoma, plasmacytoma, plasma-cell myeloma, post-transplant lymphoproliferative disorders, Waldenstrom's macroglobulinemia and anaplastic large cell lymphoma (ALCL).

In some embodiments, the immune disease comprises psoriasis, psoriatic arthritis, dermatitis, systemic scleroderma and sclerosis, inflammatory bowel disease (IBD), Crohn's disease, ulcerative colitis, respiratory distress syndrome, meningitis, encephalitis, uveitis, glomerulonephritis, eczema, asthma, atherosclerosis, leukocyte adhesion deficiency, multiple sclerosis, Raynaud's syndrome, Sjogren's syndrome, juvenile-onset diabetes, Reiter's disease, Behcet's disease, immune complex nephritis, IgA nephropathy, IgM polyneuropathy, neuromyelitis optical, immune-mediated thrombocytopenia, hemolytic anemia, myasthenia gravis, lupus nephritis, systemic lupus erythematosus, rheumatoid arthritis (RA), atopic dermatitis, pemphigus. Graves' disease. Hashimoto's thyroiditis, Wegener's granulomatosis, an Omenn's syndrome, chronic renal failure, acute infectious mononucleosis, and chronic obstructive pulmonary disease.

pH is found to have a great influence on the stability of the formulations during formulation development, so that it is necessary to maintain a specific pH range for the formulations of the present invention. The present invention selects a suitable buffer system, such as succinate buffer, citrate buffer, phosphate buffer, histidine buffer or acetate buffer, and maintains the pH value in a range from 5.5 to 6.2. The trehalose as a stabilizer can reduce aggregation and degradation of antibody proteins, thus effectively protecting the activity of active ingredients in the formulations. Polysorbate-80 as a surfactant plays an important role in preventing aggregation of antibody proteins. The antibody formulations of the present invention have a good stability.

In the fourth aspect, the present invention further provides a method or use for treating a disease, wherein the method or use comprises: administering to a patient in need thereof a composition comprising an effective amount of an anti-CD20 monoclonal antibody (mAb) or an antigen-binding fragment thereof, which has specificity to a human CD20 and comprises:
a light chain variable region (VL) comprising the amino acid sequence of SEQ ID NO: 1 or a polypeptide having at least 80% sequence identity to SEQ ID NO: 1; and
a heavy chain variant region (VH) comprising an amino acid sequence of SEQ ID NO: 2 or a polypeptide having at least 80% sequence identity to SEQ ID NO: 2; and
wherein the effective amount is about 10 mg to about 3000 mg per dose.

In some embodiments, the composition is a formulation described herein.

In some embodiments, the patient has a disease characterized by CD20 positive cells. The disease can be neuromyelitis optica spectrum disorders (NMOSD), non-Hodgkin's lymphoma (NHL), multiple sclerosis (MS), immune thrombocytopenia (ITP), rheumatoid arthritis (RA), Wegener's granulomatosis (WG), microscopic polyangiitis (MPA), lupus nephritis, systemic lupus erythematosus and chronic lymphocytic leukemia (CLL). The disease can be refractory to or relapsed after at least one prior standard therapeutic regimen.

CD20-positive B cell is a specific type of immune cell. Studies have shown that it plays a very important role in the pathogenesis of NMOSD and is considered to be a key factor leading to myelin and axonal damage. AQP4-IgG enters the CNS when the permeability of the blood-brain barrier increases, and binds to the AQP4 antigen on the astrocyte foot process, which in turn leads to down-regulation of AQP4 expression and perturbations of water homoeostasis in the CNS and eventually leads to the death of astrocytes and oligodendrocytes and even neurons, causes demyelination and neurological deficits in nerve cells. Studies have shown that AQP4-IgG can only be produced by differentiation of B cells, therefore, CD20 molecule is one of the ideal targets for the treatment of neuromyelitis optica. Studies have shown that B cell depletion therapy by monoclonal antibodies against CD20 on the surface of B cells (such as rituximab) can reduce the recurrence of NMOSD and slow the progression of neurological dysfunction, and has a significant therapeutic effect; the main mechanism for its clearance of B cells is antibody-dependent cellular cytotoxicity (ADCC), complement-dependent cytotoxicity (CDC), apoptosis, etc.

In some embodiments, the VL comprises a polypeptide having at least 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values (end points included), sequence identity to SEQ ID NO: 1. In some embodiments, the VH comprises a polypeptide having at least 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values (end points included), sequence identity to SEQ ID NO:2.

In some embodiments, the VL comprises the amino acid sequence of SEQ ID NO: 1, and the VH comprises the amino acid sequence of SEQ ID NO: 2.

In some embodiments, the anti-CD20 mAb comprises:
a light chain comprising the amino acid sequence of SEQ ID NO: 3 or a polypeptide having at least 80% sequence identity to SEQ ID NO: 3; and
a heavy chain comprising an amino acid sequence of SEQ 1D NO: 4 or a polypeptide having at least 80% sequence identity to SEQ ID NO: 4.

In some embodiments, the light chain comprises a polypeptide having at least 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values (end points included), sequence identity to SEQ ID NO: 3. In some embodiments, the heavy chain comprises a polypeptide having at least 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values (end points included), sequence identity to SEQ ID NO:4.

In some embodiments, the anti-CD20 mAb comprises:
a light chain comprising the amino acid sequence of SEQ ID NO: 3; and
a heavy chain comprising an amino acid sequence of SEQ ID NO: 4.

In some embodiments, the anti-CD20 mAb is BAT4406F which comprises:
a light chain comprising the amino acid sequence of SEQ ID NO: 3; and
a heavy chain comprising an amino acid sequence of SEQ ID NO: 4;
and is characterized with a very low (no more than 5%) fucose content.

BAT4406F is a glycosylation-optimized IgG₁ subclass of fully human monoclonal antibody described in International Patent Application PCT/CN2018/100008.

In some embodiments, the anti-CD20 mAb or the antigen-binding fragment thereof (e.g., BAT4406F) is produced from a cell line having α-(1,6)-fucosyltransferase gene knocked out, which can express antibodies having a low fucose content (e.g., 0-5%) and an enhanced ADCC effect. In some embodiments, the cell line is CHO-BAT-KF cell line described in International Patent Application PCT/CN2018/10008. The taxonomic denomination of the CHO host cell strain is Chinese hamster overy cells CHO-BAT-KF FUT8(−/−). An anti-CD20 mAb (e.g., BAT4406F) or an antigen binding fragment thereof can be expressed and purified as described in International Patent Application PCT/CN2018/100008. Purification can be carried out by conventional methods, such as centrifuging the cell suspension and harvesting the supernatant, which can be further cleared by centrifuging. Protein A affinity and ion exchange columns can be used to purify the antibodies.

In some embodiments, the glycosylation of the anti-CD20 mAb (e.g., BAT4406F) is characterized by one or more of the following:
the fucose content of the antibody is very low (e.g., 0-5%);
the galactose level of the antibody is low (e.g., ≤30%);
the mannose level of the antibody is low (e.g., ≤5%);
the high mannose level of the antibody is low (e.g., ≤5%);
the G0 level of the antibody is high (e.g., ≥60%).

In some embodiments, the antibody has a low galactose level, e.g., ≤5%.

In some embodiments, the antibody has a high G0 level, e.g., ≥80%.

In some embodiments, the glycosylation site of the antibody is an Asn residue on the heavy chain, such as Asn297 in SEQ ID NO 4.

In some embodiments, at least about 50% of the anti-CD20 mAb (e.g., BAT4406F) can comprise the G0 glycan at the asparagine residue 297 (Asn297) of SEQ ID NO: 4, and/or at most about 10% of the anti-CD20 mAb can comprise the Man5 glycan at the amino acid residue 297 of SEQ ID NO: 4. In some embodiments, less than about 20% of the anti-CD20 mAb comprise a fucosyl residue. In some embodiments, less than about 10% of the anti-CD20 mAb comprise a fucosyl residue. In some embodiments, less than about 5% of the anti-CD20 mAb comprise a fucosyl residue. In some embodiments, less than about 1% of the anti-CD20 mAb comprise a fucosyl residue.

In some embodiments, at least 50% of the anti-CD20 mAb (e.g., BAT4406F) comprises the G0 glycan at an N-glycosylation site of a constant region of the heavy chain of the anti-CD20 antibody. In some embodiments, at least 60% of the anti-CD20 mAb comprises the G0 glycan at an N-glycosylation site of a constant region of the heavy chain of the anti-CD20 antibody. In some embodiments, at least about 65% of the anti-CD20 mAb can comprise the G0 glycan at the N-glycosylation site of the constant region of the heavy chain of the anti-CD20 antibody. In some embodiments, at most 7% of the anti-CD20 mAb can comprise the Man5 glycan at the N-glycosylation site of the constant region of the heavy chain of the anti-CD20 antibody. In some embodiments, at most about 5% of the anti-CD20 mAb can comprise the Man5 glycan at the N-glycosylation site of the constant region of the heavy chain of anti-CD20 antibody. In some embodiments, at most about 3% of the anti-CD20 mAb can comprise the Man5 glycan at the N-glycosylation site of the constant region of the heavy chain of anti-CD20 antibody.

In some embodiments, the oligosaccharides of the Fc region of the anti-CD20 mAb (e.g., BAT4406F) in the composition comprise no more than about 10% of fucosyl content. In some embodiments, the oligosaccharides of the Fc region of the anti-CD20 mAb comprise no more than about 5% of fucosyl content. In some embodiments, the oligosaccharides of the Fc region of the anti-CD20 mAb comprise no more than about 1% of fucosyl content.

In some embodiments, at least, or at least about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, or a number or a range between any two of these values (end points included), of the anti-CD20 mAb in the composition can comprise the G0 glycan at the N-glycosylation site of the constant region of the heavy chain of the anti-CD20 mAb. In some embodiments, at most, or at most about, 1%, 2%, 3%, 4%, 5%, 6%, 7%, 8%, 9%, 10%, 11%, 12%, 13%, 14%, 15%, 16%, 17%, 18%, 19%, 20%, or a number or a range between any two of these values (end points included), of the anti-CD20 mAb in the composition can comprise any glycans other than the G0 glycan at the N-glycosylation site of the constant region of the heavy chain of the anti-CD20 mAb.

In some embodiments, at most, or at most about, 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, or a number or a range between any two of these values (end points included), of the anti-CD20 mAb (e.g., BAT4406F) can comprise the G0-GN glycan at the N-glycosylation site of the constant region of the heavy chain of the anti-CD20 mAb. In some embodiments, at most, or at most about, 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, or a number or a range between any two of these values (end points included), of the anti-CD20 mAb can comprise the Man5 glycan at the N-glycosylation site of the constant region of the heavy chain of the anti-CD20 mAb. In some embodiments, at most, or at most about, 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, or a number or a range between any two of these values (end points included), of the anti-CD20 mAb can comprise the G1 glycan at the N-glycosylation site of the constant region of the heavy chain of the anti-CD20 mAb. In some embodiments, at most, or at most about, 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, or a number or a range between any two of these values (end points included), of the anti-CD20 mAb can comprise the G1′ glycan at the N-glycosylation site of the constant region of the heavy chain of the anti-CD20 mAb. In some embodiments, at most, or at most about, 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, or a number or a range between any two of these values (end points included), of the anti-CD20 mAb can comprise the G2 glycan at the N-glycosylation site of the constant region of the heavy chain of the anti-CD20 mAb.

In some embodiments, the fucose content of the anti-CD20 mAb (e.g., BAT4406F) in the composition is very low. For example, the fucose content of the anti-CD20 mAb in the composition is, is about, or is at most, 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, or a number or a range between any two of these values (end points included). In some embodiments, the anti-CD20 mAb (e.g., BAT4406F) in the composition do not have any fucose residue, i.e., the composition comprises nonfucosylated or afucosylated anti-CD20 mAb (e.g., nonfucosylated or afucosylated BAT4406F).

In some embodiments, the galactose level of the anti-CD20 mAb is low. For example, the galactose level of the antibody is, is about, or is at most, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, or a number or a range between any two of these values (end points included).

In some embodiments, the mannose content of the anti-CD20 mAb is very low. For example, the mannose content of the anti-CD20 mAb is, is about, or is at most, 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, or a number or a range between any two of these values (end points included).

In some embodiments, the high mannose content of the anti-CD20 mAb is very low. For example, the mannose content of the anti-CD20 mAb is, is about, or is at most, 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, or a number or a range between any two of these values (end points included).

In some embodiments, the G0 level of the anti-CD20 mAb is high. For example, the G0 level of the anti-CD20 mAb is, is about, or is at least, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%. 86%, 87%, 88%, 89%, 90%, 91%, 92%, 93%, 94%, 95%, 96%, 97%, 98%, 99%, 99.9%. or a number or a range between any two of these values (end points included).

In some embodiments, the disease being treated by the method or use disclosed herein is a disease or disorder treatable by B-cell depletion.

In some embodiments, the disease being treated by the method or use disclosed herein is selected from the group consisting of cancers, anaphylaxis, cardiovascular diseases, inflammatory diseases, autoimmune diseases, metabolic diseases, neurological diseases, viral infections, and bacterial infections. For example, the disease may be cancer or anaphylaxis.

In some embodiments, the disease is selected from pernicious amenia (Addison's disease), amyotrophic lateral sclerosis, ankylosing spondylitis, erythema multiforme, lupus nephritis, dermatomyositis, immune-mediated thrombocytopenias, such as acute idiopathic thrombocytopenic purpurea and chronic idiopathic thrombocytopenic purpurea, Sydenham's chorea, rheumatic fever, polyglandular syndromes, Henoch-Schonlein purpura, post-streptococcal nephritis, erythema nodosum, Takayasu's arteritis, polyarteritis nodosa, Goodpasture's syndrome, thromboangitis ubiterans, primary biliary cirrhosis, Hashimoto's thyroiditis, thyrotoxicosis, chronic active hepatitis, polymyositis/dermatomyositis, polychondritis, pamphigus vulgaris, Wegener's granulomatosis, membranous nephropathy, tabes dorsalis, polymyaglia, pernicious anemia, rapidly progressive glomerulonephritis and fibrosing alveolitis, inflammatory diseases such as inflammatory skin diseases including psoriasis and dermatitis (e.g. atopic dermatitis); systemic scleroderma and sclerosis; diseases associated with inflammatory bowel disease (such as Crohn's disease and ulcerative colitis); respiratory distress syndrome (including adult respiratory distress syndrome; ARDS), dermatitis, meningitis, encephalitis, colitis, glomerulonephritis, uveitis, allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE); diabetes mellitus (e.g. Type 1 diabetes mellitus or insulin dependent diabetes mellitus); multiple sclerosis; Reynaud's syndrome; autoimmune thyroiditis; allergic encephalomyelitis; Sjorgen's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia etc.

In some embodiments, the disease is neuromyelitis optica spectrum disorders (NMOSD), non-Hodgkin's lymphoma (NHL), multiple sclerosis (MS), immune thrombocytopenia (ITP), rheumatoid arthritis (RA), Wegener's granulomatosis (WG), microscopic polyangiitis (MPA), lupus nephritis, systemic lupus erythematosus or chronic lymphocytic leukemia (CLL).

In some embodiments, the disease is refractory to or relapsed after at least one prior therapeutic regimen. In some embodiments, the disease is refractory to or relapsed after at least two prior therapeutic regimens.

In some embodiments, provided herein is a method of treating a neuromyelitis optica spectrum disorder in a patient in need thereof comprising administering an effective amount of the anti-CD20 mAb (e.g., BAT4406F) or the antigen-binding fragment thereof as described herein, wherein the effective amount is about 10 mg to about 3000 mg per dose.

In some embodiments, provided is use of an effective amount of the anti-CD20 mAb (e.g., BAT4406F) or the antigen-binding fragment thereof in the manufacture of a medicament in the treatment of a disease as described herein, wherein the effective amount is about 0.5 mg/kg to about 30 mg/kg or about 10 mg to about 3000 mg per dose.

In the fifth aspect, provided is a kit comprising the anti-CD20 mAb (e.g., BAT4406F) or the antigen-binding fragment thereof for the treatment of a disease as described herein, and instructions to administer the anti-CD20 mAb (e.g., BAT4406F) or the antigen-binding fragment thereof to a patient in need thereof in an amount described herein.

The anti-CD20 mAb (e.g., BAT4406F) or the antigen-binding fragment thereof can be formulated as pharmaceutical compositions and administered to the patient in a variety of forms adapted to the chosen route of administration, e.g., parenterally, by intravenous (iv.), intramuscular, topical or subcutaneous routes. The amount of the anti-CD20 mAb (e.g., BAT4406F) or the antigen-binding fragment thereof will vary depend on the nature of the drug, degree of cell surface triggered the internalization, trafficking, and release of the drug, the disease being treated, the conditions of the patient, such as age, gender, weight, etc.

In some embodiments, the amount of the anti-CD20 mAb (e.g., BAT4406F) or the antigen-binding fragment thereof administered each time is, or is about, 0.5 mg/kg to 20 mg/kg. For example, the amount of the anti-CD20 mAb (e.g., BAT4406F) or the antigen-binding fragment thereof administered each time can be, or can be about, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, or a number or a range between any two of these values. For example, the amount of the antibody administered each time can be about 0.5 mg/kg to 10 mg/kg. For example, the effective amount can be about 0.5 mg/kg to 0.9 mg/kg, 0.7 mg/kg to 1.3 mg/kg, 1.5 mg/kg to 2.5 mg/kg, 3 mg/kg to 5 mg/kg, 5 mg/kg to 7 mg/kg, 7 mg/kg to 9 mg/kg, or 9 mg/kg to 10 mg/kg.

In some embodiments, the anti-CD20 mAb (e.g., BAT4406F) or the antigen-binding fragment thereof can be administered, or administered about once every two weeks, once every three weeks, once every four weeks, once every five weeks, once every six weeks, once every seven weeks, once every eight weeks, once every three months, once every four months, once every five months, once every six months, or a number or a range between any two of these values. For example, the effective amount administered can be about 0.5 mg/kg to about 10 mg/kg about once every month, every two, three, four, five or six months. The effective amount administered can be about 0.5 mg/kg to 10 mg/kg about once every month, every two, three, four, five or six months, such as about 0.5 mg/kg to 0.9 mg/kg, 0.7 mg/kg to 1.3 mg/kg, 1.5 mg/kg to 2.5 mg/kg, 3 mg/kg to 5 mg/kg, 5 mg/kg to 7 mg/kg, 7 mg/kg to 9 mg/kg, or 9 mg/kg to 10 mg/kg once every month, every two, three, four, five or six months. The effective amount administered can be about 0.5 mg/kg, 1 mg/kg, 2 mg/kg, 4 mg/kg, 6 mg/kg, 8 mg/kg, or 10 mg/kg once every three weeks to 6 months.

In some embodiments, provided is a method of treating a disease, comprising administering to a patient in need thereof an effective amount of the anti-CD20 mAb (e.g., BAT4406F) or the antigen-binding fragment thereof, wherein the effective amount is about 20 mg to about 2000 mg once every three weeks to 6 months.

In the sixth aspect, provided is use of the anti-CD20 mAb (e.g., BAT4406F) or the antigen-binding fragment thereof in the manufacture of a medicament in the treatment of a disease as described herein, wherein the anti-CD20 mAb or the antigen-binding fragment thereof is for administration to a patient at an amount of about 20 mg to about 2000 mg once every three weeks to six months.

In some embodiments, the effective amount is about 50 mg to about 1000 mg once every month to six months. In some embodiments, the effective amount is about 100 mg to about 500 mg once every month, every two months, every three months, every four months, every five months, or every six months. In some embodiments, the effective amount is about 50 mg to about 300 mg once every month, every two months, every three months, every four months, every five months, or every six months. In some embodiments, the effective amount is about 100 mg to about 300 mg once every two months, or every three months. In some embodiments, the effective amount is about 100 mg, about 200 mg, or about 300 mg once every two months, or every three months.

In some embodiments, the effective amount is about 20 mg, 50 mg, 100 mg, 150 mg, 200 mg, 250 mg, 300 mg, 350 mg, 400 mg, 450 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, or 2000 mg once every month to six months. In some embodiments, the effective amount is about 20 mg, 50 mg, 100 mg, 150 mg, 200 mg, 250 mg, 300 mg, 350 mg, 400 mg, 450 mg, 500 mg, 600 mg, 700 mg, 800 mg, 900 mg, 1000 mg. 1100 mg, 1200 mg, 1300 mg, 1400 mg, 1500 mg, 1600 mg, 1700 mg, 1800 mg, 1900 mg, or 2000 mg once every month, every two months, every three months, every four months, every five months, or every six months. In some embodiments, the effective amount is about 100 mg, 150 mg, 200 mg, 250 mg, 300 mg, 350 mg, 400 mg, 450 mg, 500 mg, or 600 mg once every month, every two months, every three months, every four months, every five months, or every six months. In some embodiments, the effective amount is about 100 mg, 150 mg, 200 mg, 250 mg, or 300 mg once every month, every two months, every three months, every four months, every five months, or every six months. In some embodiments, the effective amount is about 100 mg, 150 mg, 200 mg, 250 mg, 300 mg, 350 mg, 400 mg, 450 mg, 500 mg, or 600 mg once every two months, or every three months. In some embodiments, the effective amount is about 100 mg, 150 mg, 200 mg, 250 mg, or 300 mg once every two months. In some embodiments, the effective amount is about 100 mg, 150 mg, 200 mg, 250 mg, or 300 mg once every three months.

In some embodiments, the total number of administration of the anti-CD20 mAb or the antigen-binding fragment thereof can be, or can be about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or a number or a range between any two of these values (end points included). For example, the anti-CD20 mAb or the antigen-binding fragment thereof can be administered for about four times.

In some embodiments, the duration of an administration can be, or can be about, 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, or a number or a range between any two of these values. For example, the administration can be carried out for about one hour. In some embodiments, the duration of an administration depends on the situation of an administration. In some embodiments, the duration of an infusion administration may exceed 10 hours. In some embodiments, the duration of infusion can be, or can be about 1 hour, 2 hours, 3 hours, 4 hours, 5 hours, 6 hours, 7 hours, 8 hours, 9 hours, 10 hours, 11 hours, 12 hours, 14 hours, or a number or a range between any two of these values (end points included).

In some embodiments, the anti-CD20 mAb (e.g., BAT4406F) or the antigen-binding fragment thereof is administered by I.V. infusion.

In some embodiments, the anti-CD20 mAb (e.g., BAT4406F) or the antigen-binding fragment thereof is administered in conjunction with another therapy. For example, the anti-CD20 mAb (e.g., BAT4406F) or the antigen-binding fragment thereof can be co-administered with another therapy for treating a disease as described herein, for example, an immunosuppressive agent, a glucocorticoid, a corticosteroid, an anti-malarial agent, a cytotoxic agent, an integrin antagonist, a cytokine antagonist, or a hormone.

In the seventh aspect, provided are pharmaceutical compositions comprising one or more antibodies as described herein, for example, such compositions may contain 0.1% or more of the antibody. The percentage of the antibodies may vary and may be between about 2 to about 90% of the weight of a given dosage form. The amount of the antibodies in such therapeutically useful compositions is such that an effective amount can be administered.

Examples of pharmaceutical compositions suitable for injection or infusion can include sterile aqueous solutions or dispersions in a pharmaceutically acceptable liquid carrier or vehicle, or sterile powders comprising the active ingredient which are adapted for the extemporaneous preparation of sterile injectable or infusible solutions or dispersions, optionally encapsulated in liposomes. Other forms of pharmaceutical compositions include topical formulations, such as gel, ointments, creams, lotions or transdermal patches, etc. The pharmaceutical compositions include using techniques well known to those in the art. Suitable pharmaceutically-acceptable carriers, outside those mentioned herein, are known in the art; for example, see Remington, The Science and Practice of Pharmacy, 20th Edition, 2000, Lippincott Williams & Wilkins, (Editors: Gennaro, A. R., et al.).

In the eighth aspect, provided are methods of producing a pharmaceutical composition comprising admixing the antibody as described herein and a pharmaceutically acceptable carrier. In some embodiments, provided is a method of producing a pharmaceutical formulation described herein, comprising admixing the antibody as described herein and the excipients, such as the buffer, the stabilizer and the surfactant, described herein. Methods of admixing an antibody with a pharmaceutically acceptable carrier are generally known in the art, for example, uniformly mixing the antibody with liquids or finely divided solid carriers, or both, in the required proportions, and then, if necessary, forming the resulting mixture into a desired shape. In some embodiments, the method comprises admixing an antibody aqueous solution with an aqueous solution comprising the excipients, such as the buffer, the stabilizer and the surfactant, described herein.

In some embodiments, the antibody is formulated as an injectable, for example, at a concentration of 2-50 mg/mL in an aqueous solution comprising 4-10 mg/mL, sodium chloride and/or 5-12 mg/mL sodium acetate, or alternatively at a concentration of 2-50 mg/mL in an aqueous solution comprising 5-10 mg/mL sodium chloride, 1-5 mg/mL sodium phosphate dibasic heptahydrate, 0.1-0.5 mg/mL sodium phosphate monobasic monohydrate.

Other examples of formulations of the antibody include an injectable formulation having a concentration of 2-100 mg/mL, of the antibody in an aqueous solution comprising 0.5-1.0% sodium chloride, 0.05-0.10% monobasic sodium phosphate dihydrate, 1.0-2.0% dibasic sodium phosphate dihydrate, 0.01-0.05% sodium citrate, 0.10-0.20% citric acid monohydrate, 1.0-2.0% mannitol, 0.1%-0.2 polysorbate 80, and Water for Injection, USP. Sodium hydroxide added as necessary to adjust pH.

In some embodiments, the antibody is formulated as described herein.

### BRIEF DESCRIPTION OF THE DRAWINGS

FIG. 1 shows the number of particles (particles/mL) in the samples containing different surfactants.

FIG. 2 shows the turbidity of the samples containing different surfactants after shaking.

FIG. 3 shows particles of the samples containing different surfactants after repeated freezing and thawing for five times.

### DETAILED DESCRIPTION

### Definitions

As used herein, the following definitions shall apply unless otherwise indicated.

As used herein, unless otherwise stated, the singular forms “a,” “an,” and “the” include plural reference. Thus, for example, a reference to “a compound” includes a plurality of compounds.

As used herein, “about” will be understood by persons of ordinary skill in the art and will vary to some extent depending upon the context in which it is used. In some embodiments, “about” will mean up to plus or minus 10% or plus or minus 5%, or plus or minus 1% of the particular term. “About x” includes “x”.

“Amino acid” refers to an α-amino carboxylic acid, which may be encoded by a nucleic acid directly or in a form of precursor. A single amino acid is encoded by a nucleic acid consisting of three nucleotides (so-called codons or base triplets). Each amino acid is encoded by at least one codon. Encoding the same amino acid by different codons is called “degeneracy of the genetic code”. The term “amino acid” used in the present application refers to a naturally occurring α-amino carboxylic acid, including alanine (with a three-letter code: ala, or a one-letter code: A), arginine (arg, R), asparagine (asn, N), aspartic acid (asp, D), cysteine (cys, C), glutamine (gin, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y) and valine (val, V).

“Antibody” is used in a broad sense thereof, and covers monoclonal antibodies (including full-length monoclonal antibodies), polyclonal antibodies, multi-specific antibodies (e.g., bi-specific antibodies) and antibody fragments, as long as they show the desired biological activity.

“Antibody fragment” comprises a part of a full-length antibody, which usually comprises its antigen-binding region. Examples of antibody fragments include Fab, Fab′, F(ab′)₂ and Fv fragments; diabodies; linear antibodies; single-chain antibodies; and multi-specific antibodies formed by the antibody fragments.

“Monoclonal antibody” (mAb) is an antibody prepared by identical immune cells, and the immune cells are all clones from a single parent cell. Monoclonal antibodies can have monovalent affinity because they bind to the same epitope (an antigen portion recognized by the antibody). On the contrary, polyclonal antibodies bind to multiple epitopes, and are usually secreted by several different plasma cells. A monoclonal antibody may also be modified into a bi-specific monoclonal antibody by increasing the therapeutic target of the single monoclonal antibody to two epitopes. Monoclonal antibodies can be prepared by hybridoma, recombination, transgene or other technologies known by those skilled in the art.

The amount of buffer in the present invention refers to the total amount of the buffer pair in the buffer system constituting the buffer. In some embodiments, molar concentration is used as the unit of the amount of the buffer, and its numerical value refers to the molar concentration of the buffer pair in the buffer system of the buffer. For example, when a histidine buffer consisting of L-histidine and L-histidine hydrochloride is used, the given concentration (e.g., 10 mM) of the histidine buffer is a combined concentration of L-histidine and L-histidine hydrochloride (e.g., L-histidine is 5 mM, and L-histidine hydrochloride is 5 mM; or L-histidine is 6 mM, and L-histidine hydrochloride is 4 mM; or L-histidine is 3.46 mM, and L-histidine hydrochloride is 6.54 mM, etc.).

The formulation of the present invention can be prepared with the excipients or hydrates thereof, for example, histidine hydrochloride, also known as histidine monohydrochloride, which may be anhydrous histidine hydrochloride or histidine hydrochloride hydrate, such as histidine hydrochloride monohydrate. “5 mM histidine hydrochloride” may be 5 mmol histidine hydrochloride or histidine hydrochloride hydrate dissolved in a solvent to form 1 L of solution; and 958 mg of histidine hydrochloride may comprise 958 mg of histidine hydrochloride or the corresponding amount of hydrate.

The following are some examples of the amount of anhydrous trehalose corresponding to the amount of trehalose hydrate: 1) 158.6 mM trehalose means that 158.6 mM anhydrous trehalose (158.6 mM anhydrous trehalose is equivalent to about 5.4% anhydrous trehalose) or 158.6 mM trehalose dihydrate (158.6 mM trehalose dihydrate is equivalent to about 6% trehalose dihydrate) can be added; and 2) 224.6 mM trehalose means that 224.6 mM anhydrous trehalose (224.6 mM anhydrous trehalose is equivalent to about 7.68% anhydrous trehalose) or 224.6 mM trehalose dihydrate (224.6 mM trehalose dihydrate is equivalent to about 8.5% trehalose dihydrate) can be added.

In the present invention, the concentration “%” represents a mass volume concentration in the unit of g/ml. For example, 0.9% sodium chloride solution represents that 0.9 g of sodium chloride is dissolved in the solvent to form 100 ml of solution, which means that the solution contains 0.9 g/100 ml sodium chloride.

As used herein, the term “comprising” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this disclosure.

“Patient” or “subject” refers to mammals and includes humans and non-human mammals. In some embodiments, the patient is a human. In some embodiments, the patient is a non-human mammal, such as a wild, domestic, and farm animal. In yet other embodiments, the patient is a dog, cat, mouse, rat, rabbit, guinea pig, or primate such as a cynomolgus monkey.

“Treating” or “treatment” of a disease in a patient refers to (1) preventing the disease from occurring in a patient that is predisposed or does not yet display symptoms of the disease; (2) inhibiting the disease or a symptom thereof or arresting its development; or (3) ameliorating or causing regression of the disease or a symptom thereof.

“Effective amount” is intended to mean an amount of an active compound or pharmaceutical agent that elicits the biological or medicinal response in a tissue, system, animal, individual or human that is being sought by a researcher, veterinarian, medical doctor or other clinician, which includes treating a disease.

“Administering” a composition may be accomplished by injection, infusion, parenteral, intravenous, mucosal, sublingual, intramuscular, intradermal, intranasal, intraperitoneal, intraarterial, subcutaneous absorption or by any method in combination with other known techniques. “Administering” may also be prescribing the composition with instructions to administer using one or more of the above techniques. In some embodiments, administration occurs systemically.

As used herein, the phrase “in need thereof” means that the patient has been identified as having a need for the particular method or treatment. In some embodiments, the identification can be by any means of diagnosis. For example, euromyelitis optica spectrum disorders can be diagnosed according to Appendix 1. In any of the methods and treatments described herein, the patient can be in need thereof.

In the following examples, AUC_{(0-mf)} represents the area under the concentration-time curve from time zero to time infinity, AUC₍₀₋ₜ₎ represents the area under the concentration-time curve from time zero to the last measurable timepoint, Cₘₐₓ represents maximum plasma concentration, Tₘₐₓ represents time to maximum plasma concentration, t_{1/2} represents amount of time required for plasma concentration to decrease by half, CLt represents plasma clearance, Vd represents apparent distribution volume, MRT represents mean retention time.

All publications are hereby incorporated by reference in their entirety for all purposes.

Disclosed herein in one aspect is a liquid formulation comprising an anti-CD20 antibody, which comprises the following ingredients: 10 mg/ml to 120 mg/ml anti-CD20 antibody, a buffer, a stabilizer and a surfactant, and the liquid formulation has a pH value ranging from 5 to 7.

In some embodiments, the liquid formulation comprises 10 mg/nil to 120 mg/ml anti-CD20 antibody, 10 mM to 30 mM buffer, 58 mM to 292 mM stabilizer and 0.1 mg/ml to 0.5 mg/ml surfactant, and the liquid formulation has a pH value ranging from 5.5 to 6.5.

In some embodiments, the liquid formulation comprises 15 mg/ml to 80 mg/nil anti-CD20 antibody, 10 mM to 30 mM buffer, 80 mM to 240 mM stabilizer and 0.1 mg/nil to 0.4 mg/ml surfactant, and the liquid formulation has a pH value ranging from 5.5 to 6.2.

In some embodiments, the buffer is selected from succinate buffer, citrate buffer, phosphate buffer, histidine buffer and acetate buffer.

In some embodiments, the stabilizer is selected from sucrose, trehalose, sorbitol, mannitol and methionine; and the surfactant is selected from polysorbate-80 and polysorbate-20.

In some embodiments, the concentration of the anti-CD20 antibody is about 15 mg/nil, 40 mg/ml, 60 mg/ml, 80 mg/nil (i.e., 8%), or a number or a range between any two of these values (end point values included); the concentration of the buffer is about 10 mM, 15 mM, 26 mM, 30 mM, or a number or a range between any two of these values (end point values included); the concentration of the stabilizer is about 80 mM, 140 mM, 190 mM, 240 mM, or a number or a range between any two of these values (end point values included); the concentration of the surfactant is about 0.1 mg/ml, 0.3 mg/ml, 0.4 mg/ml (i.e., 0.04%), or a number or a range between any two of these values (end point values included); and the pH value is about 5.5, 5.6, 5.7, 6.0, 6.2, or a number or a range between any two of these values (end point values included).

In some embodiments, the anti-CD20 antibody comprises:
a light chain variable region (VL) comprising the amino acid sequence of SEQ ID NO: 1 or a polypeptide having at least 80% sequence identity to SEQ ID NO: 1; and
a heavy chain variant region (VH) comprising an amino acid sequence of SEQ ID NO: 2 or a polypeptide having at least 80% sequence identity to SEQ ID NO: 2.

In some embodiments, the anti-CD20 antibody is BAT4406F.

Disclosed herein in a further aspect is a method for treating a disease involving cells expressing CD20 in a patient, comprises administering to a patient in need thereof an effective amount of the above liquid formulation.

In some embodiments, the patient is human.

In some embodiments, the patient is administrated by injection, such as intravenous or subcutaneous injection.

In some embodiments, when the patient is administrated by injection, the liquid formulation comprises the following ingredients: about 20 mg/ml anti-CD20 antibody, about 20 mM histidine buffer, about 158.6 mM trehalose and about 0.2 mg/ml polysorbate-80, and the pH value of the liquid formulation is about 5.8.

In some embodiments, when the patient is administrated by injection, the liquid formulation comprises the following ingredients: about 20 mg/ml anti-CD20 antibody, about 20 mM histidine buffer, about 224.6 mM trehalose and about 0.2 mg/ml polysorbate-80, and the pH value of the liquid formulation is about 5.8.

In some embodiments, when the patient is administrated by injection, the liquid formulation comprises the following ingredients: about 50 mg/ml anti-CD20 antibody, about 20 mM histidine buffer, about 224.6 mM trehalose and about 0.2 mg/ml polysorbate-80, and the pH value of the liquid formulation is about 5.8.

In some embodiments, the disease involving cells expressing CD20 is selected from a tumorigenic disease and an immune disease.

In some embodiments, the tumorigenic disease comprises B-cell lymphoma, the B-cell lymphoma comprises precursor B-cell lymphocytic leukemia/lymphoma and mature B-cell tumor, lymphoplasmacytic lymphoma, mantle cell lymphoma (MCL), low-grade, moderate-grade and high-grade follicular lymphomas, cutaneous follicle center lymphoma, MALT-type, nodular-type and splenic-type marginal zone B-cell lymphomas, hairy cell leukemia, diffuse large B-cell lymphoma, Burkitt lymphoma, plasmacytoma, plasma-cell myeloma, post-transplant lymphoproliferative disorders, Waldenstrom's macroglobulinemia and anaplastic large cell lymphoma (ALCL).

In some embodiments, the tumorigenic disease comprises immune disease, the immune disease comprises psoriasis, psoriatic arthritis, dermatitis, systemic scleroderma and sclerosis, inflammatory bowel disease (IBD), Crohn's disease, ulcerative colitis, respiratory distress syndrome, meningitis, encephalitis, uveitis, glomerulonephritis, eczema, asthma, atherosclerosis, leukocyte adhesion deficiency, multiple sclerosis, Raynaud's syndrome, Sjogren's syndrome, juvenile-onset diabetes, Reiter's disease, Behcet's disease, immune complex nephritis, IgA nephropathy, IgM polyneuropathy, neuromyelitis optica, immune-mediated thrombocytopenia, hemolytic anemia, myasthenia gravis, lupus nephritis, systemic lupus erythematosus, rheumatoid arthritis (RA), atopic dermatitis, pemphigus, Graves' disease, Hashimoto's thyroiditis, Wegener's granulomatosis, Omenn's syndrome, chronic renal failure, acute infectious mononucleosis, and chronic obstructive pulmonary disease.

In some embodiments, the disease involving cells expressing CD20 is a disease or disorder treatable by B-cell depletion.

In some embodiments, the disease or disorder treatable by B-cell depletion is selected from the group consisting of cancers, anaphylaxis, cardiovascular diseases, inflammatory diseases, autoimmune diseases, metabolic diseases, neurological diseases, viral infections, and bacterial infections. For example, the disease may be cancer or anaphylaxis.

In some embodiments, the disease is selected from pernicious amenia (Addison's disease), amyotrophic lateral sclerosis, ankylosing spondylitis, erythema multiforme, lupus nephritis, dermatomyositis, immune-mediated thrombocytopenias, such as acute idiopathic thrombocytopenic purpurea and chronic idiopathic thrombocytopenic purpurea, Sydenham's chorea, rheumatic fever, polyglandular syndromes, Henoch-Schonlein purpura, post-streptococcal nephritis, erythema nodosum, Takayasu's arteritis, polyarteritis nodosa, Goodpasture's syndrome, thromboangitis ubiterans, primary biliary cirrhosis, Hashimoto's thyroiditis, thyrotoxicosis, chronic active hepatitis, polymyositis/dermatomyositis, polychondritis, pamphigus vulgaris, Wegener's granulomatosis, membranous nephropathy, tabes dorsalis, polymyaglia, pernicious anemia, rapidly progressive glomerulonephritis and fibrosing alveolitis, inflammatory diseases such as inflammatory skin diseases including psoriasis and dermatitis (e.g. atopic dermatitis); systemic scleroderma and sclerosis; diseases associated with inflammatory bowel disease (such as Crohn's disease and ulcerative colitis); respiratory distress syndrome (including adult respiratory distress syndrome; ARDS), dermatitis, meningitis, encephalitis, colitis, glomerulonephritis, uveitis, allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE); diabetes mellitus (e.g. Type 1 diabetes mellitus or insulin dependent diabetes mellitus); multiple sclerosis; Reynaud's syndrome; autoimmune thyroiditis; allergic encephalomyelitis; Sjorgen's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lan-then-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia etc.

In some embodiments, the disease is neuromyelitis optica spectrum disorders (NMOSD), non-Hodgkin's lymphoma (NHL), multiple sclerosis (MS), immune thrombocytopenia (ITP), rheumatoid arthritis (RA), Wegener's granulomatosis (WG), microscopic polyangiitis (MPA), lupus nephritis, systemic lupus erythematosus or chronic lymphocytic leukemia (CLL).

In some embodiments, the disease is refractory to or relapsed after at least one prior therapeutic regimen. In some embodiments, the disease is refractory to or relapsed after at least two prior therapeutic regimens.

Disclosed herein in a further aspect is use of the above liquid formulation for the formulation of a medicament for treating a disease involving cells expressing CD20 in a patient, wherein the disease involving cells expressing CD20 is selected from tumorigenic disease and immune disease.

In some embodiments, the patient is human.

In some embodiments, the tumorigenic disease comprises B-cell lymphoma, the B-cell lymphoma comprises precursor B-cell lymphocytic leukemia/lymphoma and mature B-cell tumor, lymphoplasmacytic lymphoma, mantle cell lymphoma (MCL), low-grade, moderate-grade and high-grade follicular lymphomas, cutaneous follicle center lymphoma, MALT-type, nodular-type and splenic-type marginal zone B-cell lymphomas, hairy cell leukemia, diffuse large B-cell lymphoma, Burkitt lymphoma, plasmacytoma, plasma-cell myeloma, post-transplant lymphoproliferative disorders, Waldenstrom's macroglobulinemia and anaplastic large cell lymphoma (ALCL).

In some embodiments, the tumorigenic disease comprises immune disease, the immune disease comprises psoriasis, psoriatic arthritis, dermatitis, systemic scleroderma and sclerosis, inflammatory bowel disease (IBD), Crohn's disease, ulcerative colitis, respiratory distress syndrome, meningitis, encephalitis, uveitis, glomerulonephritis, eczema, asthma, atherosclerosis, leukocyte adhesion deficiency, multiple sclerosis, Raynaud's syndrome, Sjogren's syndrome, juvenile-onset diabetes, Reiter's disease, Behcet's disease, immune complex nephritis, IgA nephropathy, IgM polyneuropathy, neuromyelitis optical, immune-mediated thrombocytopenia, hemolytic anemia, myasthenia gravis, lupus nephritis, systemic lupus erythematosus, rheumatoid arthritis (RA), atopic dermatitis, pemphigus, Graves' disease, Hashimoto's thyroiditis, Wegener's granulomatosis, an Omenn's syndrome, chronic renal failure, acute infectious mononucleosis, and chronic obstructive pulmonary disease.

Disclosed herein in a further aspect is a method or use for treating a disease. In some embodiments, the method or use comprises: administering to a patient in need thereof a composition comprising an effective amount of an anti-CD20 monoclonal antibody (mAb) or an antigen-binding fragment thereof, which has specificity to a human CD20 and comprises:
a light chain variable region (VL) comprising the amino acid sequence of SEQ ID NO: 1 or a polypeptide having at least 80% sequence identity to SEQ ID NO: 1; and
a heavy chain variant region (VH) comprising an amino acid sequence of SEQ ID NO: 2 or a polypeptide having at least 80% sequence identity to SEQ ID NO: 2; and
wherein the effective amount is about 10 mg to about 3000 mg per dose.

In some embodiments, the anti-CD20 monoclonal antibody (mAb) is BAT4406F.

In some embodiments, the disease being treated by the method or use disclosed herein is a disease or disorder treatable by B-cell depletion.

In some embodiments, the disease being treated by the method or use disclosed herein is selected from the group consisting of cancers, anaphylaxis, cardiovascular diseases, inflammatory diseases, autoimmune diseases, metabolic diseases, neurological diseases, viral infections, and bacterial infections. For example, the disease may be cancer or anaphylaxis.

In some embodiments, the disease is selected from pernicious amenia (Addison's disease), amyotrophic lateral sclerosis, ankylosing spondylitis, erythema multiforme, lupus nephritis, dermatomyositis, immune-mediated thrombocytopenias, such as acute idiopathic thrombocytopenic purpurea and chronic idiopathic thrombocytopenic purpurea, Sydenham's chorea, rheumatic fever, polyglandular syndromes, Henoch-Schonlein purpura, post-streptococcal nephritis, erythema nodosum, Takayasu's arteritis, polyarteritis nodosa, Goodpasture's syndrome, thromboangitis ubiterans, primary biliary cirrhosis, Hashimoto's thyroiditis, thyrotoxicosis, chronic active hepatitis, polymyositis/dermatomyositis, polychondritis, pamphigus vulgaris, Wegener's granulomatosis, membranous nephropathy, tabes dorsalis, polymyaglia, pernicious anemia, rapidly progressive glomerulonephritis and fibrosing alveolitis, inflammatory diseases such as inflammatory skin diseases including psoriasis and dermatitis (e.g. atopic dermatitis); systemic scleroderma and sclerosis; diseases associated with inflammatory bowel disease (such as Crohn's disease and ulcerative colitis); respiratory distress syndrome (including adult respiratory distress syndrome; ARDS), dermatitis, meningitis, encephalitis, colitis, glomerulonephritis, uveitis, allergic conditions such as eczema and asthma and other conditions involving infiltration of T cells and chronic inflammatory responses: atherosclerosis; leukocyte adhesion deficiency; rheumatoid arthritis; systemic lupus erythematosus (SLE); diabetes mellitus (e.g. Type 1 diabetes mellitus or insulin dependent diabetes mellitus); multiple sclerosis; Reynaud's syndrome; autoimmune thyroiditis; allergic encephalomyelitis; Sjorgen's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia etc.

In some embodiments, the disease is neuromyelitis optica spectrum disorders (NMOSD), non-Hodgkin's lymphoma (NHL), multiple sclerosis (MS), immune thrombocytopenia (ITP), rheumatoid arthritis (RA), Wegener's granulomatosis (WG), microscopic polyangiitis (MPA), lupus nephritis, systemic lupus erythematosus or chronic lymphocytic leukemia (CLL).

In some embodiments, the disease is neuromyelitis optica spectrum disorders (NMOSD), including neuromyelitis optica (NMO).

In some embodiments, the NMOSD is characterized by optic neuritis (ON) and longitudinally extensive transverse myelitis (LETM). In some embodiments, the neuromyelitis optica spectrum disorders (NMOSD) is diagnosed according to the NMOSD diagnostic criteria established by the International Panel for NMO Diagnosis (IPND), e.g., in Appendix 1. In some embodiments, the NMOSD is AQP4-IgG positive. In some embodiments, the NMOSD is AQP4-IgG negative or unknown.

In some embodiments, the NMOSD is characterized by optic neuritis. In some embodiments, the NMOSD is characterized by acute myelitis. In some embodiments, the NMOSD is characterized by area postrema syndrome (APS). In some embodiments, the NMOSD is characterized by acute brain stem syndrome. In some embodiments, the NMOSD is characterized by symptomatic narcolepsy. In some embodiments, the NMOSD is characterized by acute diencephalic clinical syndrome. In some embodiments, the NMOSD is characterized by symptomatic cerebral syndrome.

In some embodiments, provided herein is a method of treating a neuromyelitis optica spectrum disorder in a patient in need thereof comprising administering an effective amount of the anti-CD20 mAb (e.g., BAT4406F) or the antigen-binding fragment thereof as described herein, wherein the effective amount is about 20 mg to about 2000 mg per single treatment cycle or multiple treatment cycles. In some embodiments, provided herein is a method of treating a neuromyelitis optica spectrum disorder in a patient in need thereof comprising administering an effective amount of the anti-CD20 mAb (e.g., BAT4406F) or the antigen-binding fragment thereof as described herein, wherein the effective amount is about 300 mg to about 2000 mg per single treatment cycle or multiple treatment cycles. Each treatment cycle can be administered by one administration (such as one injection) or by several administrations (such as 2, 3, 4 or 5 injections) spread out during period of the treatment cycle, such as one week, two weeks, three weeks, four weeks or a month.

In some embodiments, the disease is neuromyelitis optica spectrum disorders (NMOSD), including neuromyelitis optica (NMO).

In some embodiments, the NMOSD is characterized by optic neuritis (ON) and longitudinally extensive transverse myelitis (LETM). In some embodiments, the neuromyelitis optica spectrum disorders (NMOSD) is diagnosed according to the NMOSD diagnostic criteria established by the International Panel for NMO Diagnosis (IPND), e.g., in Appendix 1. In some embodiments, the NMOSD is AQP4-IgG positive. In some embodiments, the NMOSD is AQP4-IgG negative or unknown.

In some embodiments, the NMOSD is characterized by optic neuritis. In some embodiments, the NMOSD is characterized by acute myelitis. In some embodiments, the NMOSD is characterized by area postrema syndrome (APS). In some embodiments, the NMOSD is characterized by acute brain stem syndrome. In some embodiments, the NMOSD is characterized by symptomatic narcolepsy. In some embodiments, the NMOSD is characterized by acute diencephalic clinical syndrome. In some embodiments, the NMOSD is characterized by symptomatic cerebral syndrome.

In some embodiments, provided herein is a method of treating a neuromyelitis optica spectrum disorder in a patient in need thereof comprising administering an effective amount of the anti-CD20 mAb (e.g., BAT4406F) or the antigen-binding fragment thereof as described herein, wherein the effective amount is about 20 mg to 2000 mg per single treatment cycle or multiple treatment cycles. In some embodiments, provided herein is a method of treating a neuromyelitis optica spectrum disorder in a patient in need thereof comprising administering an effective amount of the anti-CD20 mAb (e.g., BAT4406F) or the antigen-binding fragment thereof as described herein, wherein the effective amount is about 300 mg to 2000 mg per single treatment cycle or multiple treatment cycles. Each treatment cycle can be administered by one administration (such as one injection) or by several administrations (such as 2, 3, 4 or 5 injections) spread out during the period of the treatment cycle, such as one week, two weeks, three weeks, four weeks or a month. In some embodiments, the disease is refractory to or relapsed after at least one prior therapeutic regimen. In some embodiments, the disease is refractory to or relapsed after at least two prior therapeutic regimens. In some embodiments, the prior therapeutic regimen comprises a standard therapeutic regimen were used to treat acute attacks, such as a corticosteroid (e.g., methylprednisolone) and plasma exchange. In some embodiments, the prior therapeutic regimen comprises a standard therapeutic regimen used to prevent attack, such as Eculizumab (a humanized mAb), and other immunosuppressive agents (e.g. azathioprine, rituximab, and mycophenolate mofetil).

The anti-CD20 mAb (e.g., BAT4406F) or the antigen-binding fragment thereof can be formulated as pharmaceutical compositions and administered to the patient in a variety of forms adapted to the chosen route of administration, e.g., parenterally, by intravenous (i.v.), intramuscular, topical or subcutaneous routes. The amount of the anti-CD20 mAb (e.g., BAT4406F) or the antigen-binding fragment thereof will vary depend on the nature of the drug, degree of cell surface triggered the internalization, trafficking, and release of the drug, the disease being treated, the conditions of the patient, such as age, gender, weight, etc.

In some embodiments, the amount of the anti-CD20 mAb (e.g., BAT4406F) or the antigen-binding fragment thereof administered each time is, or is about, 5 mg/kg to 30 mg/kg. For example, the amount of the anti-CD20 mAb (e.g., BAT4406F) or the antigen-binding fragment thereof administered each time can be, or can be about, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg. 23 mg/kg, 24 mg/kg, 25 mg/kg, 26 mg/kg, 27 mg/kg, 28 mg/kg, 29 mg/kg, 30 mg/kg, or a number or a range between any two of these values (end points included).

In some embodiments, provided is a method of treating NMOSD, comprising administering to a patient in need thereof an effective amount of the anti-CD20 mAb (e.g., BAT4406F) or the antigen-binding fragment thereof, wherein the effective amount is ranging from 20 mg to 2000 mg per single treatment cycle or multiple treatment cycles. In some embodiments, provided is a method of treating NMOSD, comprising administering to a patient in need thereof an effective amount of the anti-CD20 mAb (e.g., BAT4406F) or the antigen-binding fragment thereof, wherein the effective amount is ranging from 300 mg to 2000 mg single treatment cycle or multiple treatment cycles. The dose schedule and mode of administration depend on the anti-CD20 mAb (e.g., BAT4406F) benefit-risk evaluation and general clinical practice guidelines in the certain disease patient population. In some embodiments, the effective amount is about 20 mg to about 2000 mg of the anti-CD20 mAb (e.g., BAT4406F) per treatment cycle, wherein a treatment cycle is administered once every month to twelve months. In some embodiments, the effective amount is about 20 mg to about 300 mg of the anti-CD20 mAb (e.g., BAT4406F) per treatment cycle, wherein a treatment cycle is administered once every month to twelve months. In some embodiments, the effective amount is about 300 mg to about 2000 mg of the anti-CD20 mAb (e.g., BAT4406F) per treatment cycle, wherein a treatment cycle is administered once every month to twelve months. In some embodiments, the effective amount is about 300 mg to about 2000 mg per treatment cycle, wherein a treatment cycle is administered once every month, every two months, every three months, every four months, every five months, every six months, every seven months, every eight months, every nine months, every ten months, every eleven months, or every twelve months. In some embodiments, the effective amount is about 500 mg to about 2000 mg per treatment cycle, wherein a treatment cycle is administered once every month, every two months, every three months, every four months, every five months, every six months, every seven months, every eight months, every nine months, every ten months, every eleven months, or every twelve months. In some embodiments, the effective amount is about 300 mg, about 500 mg, about 1000 mg, about 1500 mg or about 2000 mg, or any range between any two of the values (end point included), per treatment cycle, wherein a treatment cycle is administered once every two months, every three months, every six months, or every twelve months.

In some embodiments, the effective amount is about 20 mg, 50 mg, 100 mg, 150 mg, 200 mg, 260 mg, 300 mg, 350 mg, 400 mg, 450 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, or 2000 mg per treatment cycle, wherein a treatment cycle is administered once every month to twelve months. In some embodiments, the effective amount is about 20 mg, 50 mg, 100 mg, 150 mg, 200 mg, 260 mg, 300 mg, 350 mg, 400 mg, 450 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, or 2000 mg per treatment cycle, wherein a treatment cycle is administered once every month, every two months, every three months, every four months, every five months, every six months, every seven months, every eight months, every nine months, every ten months, every eleven months, or every twelve months. In some embodiments, the effective amount is about 20 mg, 50 mg, 100 mg, 150 mg, 200 mg, 260 mg, 300 mg, 350 mg, 400 mg, 450 mg, 500 mg, or 600 mg per treatment cycle, wherein a treatment cycle is administered once every month, every two months, every three months, every four months, every five months, every six months, every seven months, every eight months, every nine months, every ten months, every eleven months, or every twelve months. In some embodiments, the effective amount is about 300 mg per treatment cycle, wherein a treatment cycle is administered once every month, every two months, every three months, every four months, every five months, every six months, every seven months, every eight months, every nine months, every ten months, every eleven months, or every twelve months. In some embodiments, the effective amount is about 20 mg, 50 mg, 100 mg, 150 mg, 200 mg, 260 mg, 300 mg, 350 mg, 400 mg, 450 mg, 500 mg, or 600 mg per treatment cycle, wherein a treatment cycle is administered once every two months, or every three months. In some embodiments, the effective amount is about 300 mg per treatment cycle, wherein a treatment cycle is administered once every two months. In some embodiments, the effective amount is about 300 mg, or a range between any two of these values (endpoints included), per treatment cycle, wherein a treatment cycle is administered once every three months. In some embodiments, the effective amount is about 500 mg, 1000 mg, 1500 mg, or 2000 mg, or a range between any two of these values (end points included), per treatment cycle, wherein a treatment cycle is administered once every six months or every twelve months.

In some embodiments, the effective amount is about 20 mg to about 2000 mg per treatment cycle of the anti-CD20 mAb (e.g., BAT4406F) once every four months. In some embodiments, the effective amount is about 20 mg to about 300 mg per treatment cycle of the anti-CD20 mAb (e.g., BAT4406F) once every four months. In some embodiments, the effective amount is about 300 mg to about 2000 mg per treatment cycle of the anti-CD20 mAb (e.g., BAT4406F) once every four months. In some embodiments, the effective amount is about 500 mg to about 1000 mg per treatment cycle, wherein a treatment cycle is administered once every four months. In some embodiments, the effective amount is about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg, or any range between any two of the values (end point included), per treatment cycle, wherein a treatment cycle is administered once every four months.

In some embodiments, the effective amount is about 20 mg to about 2000 mg of the anti-CD20 mAb (e.g., BAT4406F) per treatment cycle, wherein a treatment cycle is administered once every five months. In some embodiments, the effective amount is about 20 mg to about 300 mg of the anti-CD20 mAb (e.g., BAT4406F) per treatment cycle, wherein a treatment cycle is administered once every five months. In some embodiments, the effective amount is about 300 mg to about 2000 mg of the anti-CD20 mAb (e.g., BAT4406F) per treatment cycle, wherein a treatment cycle is administered once every five months. In some embodiments, the effective amount is about 500 mg to about 1000 mg per treatment cycle, wherein a treatment cycle is administered once every five months. In some embodiments, the effective amount is about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg, or any range between any two of the values (end point included), per treatment cycle, wherein a treatment cycle is administered once every five months.

In some embodiments, the effective amount is about 20 mg to about 2000 mg of the anti-CD20 mAb (e.g., BAT4406F) per treatment cycle, wherein a treatment cycle is administered once every six months. In some embodiments, the effective amount is about 20 mg to about 300 mg of the anti-CD20 mAb (e.g., BAT4406F) per treatment cycle, wherein a treatment cycle is administered once every six months. In some embodiments, the effective amount is about 300 mg to about 2000 mg of the anti-CD20 mAb (e.g., BAT4406F) per treatment cycle, wherein a treatment cycle is administered once every six months. In some embodiments, the effective amount is about 500 mg to about 1000 mg per treatment cycle, wherein a treatment cycle is administered once every six months. In some embodiments, the effective amount is about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg, or any range between any two of the values (end point included), per treatment cycle, wherein a treatment cycle is administered once every six months.

In some embodiments, the effective amount is about 20 mg to about 2000 mg of the anti-CD20 mAb (e.g., BAT4406F) per treatment cycle, wherein a treatment cycle is administered once every twelve months. In some embodiments, the effective amount is about 20 mg to about 300 mg of the anti-CD20 mAb (e.g., BAT4406F) per treatment cycle, wherein a treatment cycle is administered once every twelve months. In some embodiments, the effective amount is about 300 mg to about 2000 mg of the anti-CD20 mAb (e.g., BAT4406F) per treatment cycle, wherein a treatment cycle is administered once every twelve months. In some embodiments, the effective amount is about 500 mg to about 1000 mg per treatment cycle, wherein a treatment cycle is administered once every twelve months. In some embodiments, the effective amount is about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg, or any range between any two of the values (end point included), per treatment cycle, wherein a treatment cycle is administered once every twelve months.

In some embodiments, the treatment cycle is administered once. In some embodiments, the treatment cycle is administered multiple times, such as 2, 3, 4, 5, 6, 7, 8, 9, or 10 times, or a range between any two of these values. For example, the treatment cycle can be administered for only once or four times.

In some embodiments, the amount of the treatment cycle is about 20 mg to about 2000 mg, wherein the treatment cycle is administered once and not repeated until as needed due to disease reoccurrence. In some embodiments, the amount of the treatment cycle is about 20 mg to about 300 mg, wherein the treatment cycle is administered once and not repeated until as needed due to disease reoccurrence. In some embodiments, the amount of the treatment cycle is about 300 mg to about 2000 mg, wherein the treatment cycle is administered once and not repeated until as needed due to disease reoccurrence. In some embodiments, the amount of the treatment cycle is about 300 mg, about 500 mg, about 1000 mg, about 1500 mg, about 2000 mg, about 2500 mg, or about 3000 mg, or any range between any two of the numbers, end points included. In some embodiments, the amount of the treatment cycle is about 500 mg. In some embodiments, the amount of the treatment cycle is about 1000 mg. In some embodiments, the amount of the treatment cycle is about 1500 mg. In some embodiments, the amount of the treatment cycle is about 2000 mg. In some embodiments, there is no disease reoccurrence until at least about 3 months after the administration. In some embodiments, there is no disease reoccurrence until at least about 6 months after the administration. In some embodiments, there is no disease reoccurrence until at least about 9 months after the administration. In some embodiments, there is no disease reoccurrence until at least about 12 months after the administration. In some embodiments, there is no disease reoccurrence after the administration. Disease reoccurrence includes a reoccurrence of a symptom of the disease, a diagnosis of the disease according to conventional diagnostic criteria for the disease, or a detection of the occurrence or potential occurrence of the disease by detection of one or more biomarker levels indicating the occurrence or potential occurrence of the disease (e.g., total T cell, total B cell, naïve B cell, NK cell, CD3⁺, CD4⁺, CD8⁺, CD19⁺, CD20⁺, CD27⁺ and/or CD40⁺ levels, etc.).

In some embodiments, the amount of the treatment cycle is administered by one injection (e.g., i.v. infusion or s.c. injection). In some embodiments, the amount of the treatment cycle is about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg, or any range between any two of the values (end point included), per treatment cycle, which is administered by one injection, and the treatment cycle is repeated every 6 months, every 12 months or as needed due to disease reoccurrence.

In some embodiments, the amount of the treatment cycle is administered by two injections (e.g., i.v. infusions or s.c. injections) made about one week, about two weeks, about three weeks or about four weeks apart. In some embodiments, the amount of the treatment cycle is administered by four injections (e.g., i.v. infusions or s.c. injections), each administered every week for four consecutive weeks. In some embodiments, the amount of the treatment cycle is administered by five injections (e.g., i.v. infusions or s.c. injections), each administered every week for five consecutive weeks.

In some embodiments, the amount of the treatment cycle is about 300 mg and is administered by a first injection of about 150 mg and a second injection of about 150 mg two weeks after the first injection. In some embodiments, the amount of the treatment cycle is about 300 mg and is administered by three injections of about 100 mg each administered every week for three consecutive weeks. In some embodiments, the treatment cycle is repeated every 6 months, every 12 months or as needed due to disease reoccurrence.

In some embodiments, the amount of the treatment cycle is about 500 mg and is administered by two injections. In some embodiments, the amount of the treatment cycle is administered by a first injection of about 250 mg and a second injection of about 250 mg two weeks after the first injection. In some embodiments, the treatment cycle is repeated every 6 months, every 12 months or as needed due to disease reoccurrence.

In some embodiments, the amount of the treatment cycle is about 1000 mg and is administered by two injections. In some embodiments, the amount of the treatment cycle is administered by a first injection of about 500 mg and a second injection of about 500 mg two weeks after the first injection. In some embodiments, the treatment cycle is repeated every 6 months, every 12 months or as needed due to disease reoccurrence.

In some embodiments, the amount of the treatment cycle is about 2000 mg and is administered by two injections. In some embodiments, the amount of the treatment cycle is administered by a first injection of about 1000 mg and a second injection of about 1000 mg two weeks after the first injection. In some embodiments, the treatment cycle is repeated every 6 months, every 12 months or as needed due to disease reoccurrence.

In some embodiments, the amount of the treatment cycle is about 400 mg and is administered by four injections (such as about 100 mg each injection). In some embodiments, each injection is administered every week for four consecutive weeks. In some embodiments, the treatment cycle is repeated every 6 months, every 12 months or as needed due to disease reoccurrence.

In some embodiments, the amount of the treatment cycle is about 500 mg and is administered by four injections (such as about 125 mg each injection). In some embodiments, each injection is administered every week for four consecutive weeks. In some embodiments, the amount of the treatment cycle is about 500 mg and is administered by five injections (such as about 100 mg each injection). In some embodiments, each injection administered every week for five consecutive weeks. In some embodiments, the treatment cycle is repeated every 6 months, every 12 months or as needed due to disease reoccurrence.

In some embodiments, the amount of the treatment cycle is about 1000 mg and is administered by four injections (such as about 250 mg each injection). In some embodiments, each injection is administered every week for four consecutive weeks. In some embodiments, the amount of the treatment cycle is about 1000 mg and is administered by five injections (such as about 200 mg each injection). In some embodiments, each injection is administered every week for five consecutive weeks. In some embodiments, the treatment cycle is repeated every 6 months, every 12 months or as needed due to disease reoccurrence.

In some embodiments, the amount of the treatment cycle is about 2000 mg and is administered by four injections (such as about 500 mg each injection). In some embodiments, each injection administered every week for four consecutive weeks. In some embodiments, the amount of the treatment cycle is about 2000 mg and is administered by five injections (such as about 400 mg each injection). In some embodiments, each injection is administered every week for five consecutive weeks. In some embodiments, the treatment cycle is repeated every 6 months, every 12 months or as needed due to disease reoccurrence.

In some embodiments, the anti-CD20 mAb BAT4406F) or the antigen-binding fragment thereof is administered by injections such as subcutaneous (s.c.) injections, intraperitoneal (i.p.) injections, parenteral injections, intraarterial injections, intravenous (i.v.) injections or intravenous (i.v.) infusions. In some embodiments, the anti-CD20 mAb (e.g., BAT4406F) or the antigen-binding fragment thereof is administered by i.v. infusion. In some embodiments, the anti-CD20 mAb (e.g., BAT4406F) or the antigen-binding fragment thereof is administered by s.c. injection.

In some embodiments, the anti-CD20 mAb (e.g., BAT4406F) or the antigen-binding fragment thereof is administered by intravenous (i.v.) infusions. In some embodiments, the duration of an i.v. infustion administration depends on situation of the administration. In some embodiments, the duration of an i.v. infusion administration is, or is about, 1 hour, 2 hours, 3 hours, 4 hours, 5 hours, 6 hours, 7 hours, 8 hours, 9 hours, 10 hours, 11 hours, 12 hours, 14 hours, or a number or a range between any two of these values end points included). For example, the administration can be carried out for about one hour.

In some embodiments, the anti-CD20 mAb (e.g., BAT4406F) or the antigen-binding fragment thereof is administered in conjunction with another therapy. For example, the anti-CD20 mAb (e.g., BAT4406F) or the antigen-binding fragment thereof can be co-administered with another therapy for treating a disease as described herein, for example, an immunosuppressive agent, a glucocorticoid, a corticosteroid, an anti-malarial agent, a cytotoxic agent, an integrin antagonist, a cytokine antagonist, or a hormone.

The technical solutions of the present invention are further illustrated hereinafter by specific examples, but the specific examples do not represent a limitation on the protection scope of the present invention. Some non-essential modifications and adjustments made by others according to the concept of the present invention still fall within the protection scope of the present invention.

Unless otherwise specified, the solvent of the liquid formulation in the following examples is water, such as water for injection, or normal saline for injection.

### EXAMPLES

### Example 1

In the following examples, the anti-CD20 antibody is BAT4406F comprising a heavy chain as shown in SEQ ID NO: 4 and a light chain as shown in SEQ ID NO: 3.

SEQ ID NO: 4
EVQLVESGGGLVQPGRSLRLSCAASGFTFNDYAMHWVRQAPGKGLEWVS
TISWNSGSIGYADSVKGRFTISRDNAKKSLYLQMNSLRAEDTALYYCAK
DIQYGNYYYGMDVWGQGTTVTVSSASTKGPSVFPLAPGSSKSTSGTAAL
GCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHY
TQKSLSLSPGK,;
SEQ ID NO: 3
EIVLTQSPATLSLSPGERATLSCRASQSVSSYLAWYQQKPGQAPRLLIY
DASNRATGIPARFSGSGSGTDFTLTISSLEPEDFAVYYCQQRSNWPITF
GQGTRLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKS FNRGEC,.

Screening of pH Range

Two buffers with a pH ranging from 5.0 to 6.5, 20 mM histidine buffer (His) and 20 mM acetate buffer (NaAc), were selected. Purified BAT4406F (the heavy chain of the antibody was as shown in SEQ ID NO: 4 and the light chain of the antibody was as shown in SEQ ID NO: 3) was ultra-filtered and the solvent was exchanged to prepare six formulations with buffers and pH values of His 5.5, His 6.0, His 6.5, NaAc 5.0. NaAc 5.5 and NaAc 6.0, and the antibody concentration was 18 mg/mL. The specific ingredients are shown in Table 1.

Samples of the formulations were subjected to a high temperature test at 40° C., analyzed on day 0 (0d), week 1 (1W), week 2 (2W) and week 3 (3W) by SEC-HPLC and IEC-HPLC. The results are shown in Table 2.

TABLE 1
Buffer composition

| Buffer ingredient | pH | Abbreviation |
|---|---|---|
| Acetic acid/sodium acetate | 5.0 | NaAc 5.0 |
| Acetic acid/sodium acetate | 5.5 | NaAc 5.5 |
| Acetic acid/sodium acetate | 6.0 | NaAc 6.0 |
| Histidine/histidine hydrochloride | 5.5 | His 5.5 |
| Histidine/histidine hydrochloride | 6.0 | His 6.0 |
| Histidine/histidine hydrochloride | 6.5 | His 6.5 |

TABLE 2
Changes in SEC and IEC of samples of different pH values over time at 40° C.

| Testing item | | | SEC-HPLC | | | IEC-HPLC | | |
|---|---|---|---|---|---|---|---|---|
| Sample | Sampling | | | Monomer | | Acidic | Main | Basic |
| name | day | | Polymer | purity | Fragment | region | peak | region |
| NaAc 5.0 | 0 | d | 0.63 | 99.37 | — | 18.95 | 77.92 | 3.13 |
| | 1 | W | 0.59 | 98.74 | 0.67 | 23.92 | 71.89 | 4.19 |
| | 2 | W | 0.51 | 97.54 | 1.94 | 31.86 | 61.44 | 6.70 |
| | 3 | W | 0.72 | 96.53 | 2.75 | 38.44 | 56.24 | 5.32 |
| NaAc 5.5 | 0 | d | 0.63 | 99.37 | — | 18.11 | 78.53 | 3.37 |
| | 1 | W | 0.64 | 98.79 | 0.57 | 23.65 | 72.32 | 4.04 |
| | 2 | W | 0.55 | 97.58 | 1.86 | 30.87 | 63.49 | 5.65 |
| | 3 | W | 0.80 | 96.50 | 2.70 | 36.94 | 58.67 | 4.39 |
| NaAc 6.0 | 0 | d | 0.69 | 99.31 | — | 18.39 | 78.25 | 3.36 |
| | 1 | W | 0.70 | 98.67 | 0.62 | 22.96 | 73.57 | 3.48 |
| | 2 | W | 0.70 | 97.49 | 1.8 | 29.96 | 65.07 | 4.97 |
| | 3 | W | 0.97 | 96.12 | 2.91 | 35.76 | 60.64. | 3.60 |
| His5.5 | 0 | d | 0.62 | 99.38 | — | 18.52 | 78.31 | 3.16 |
| | 1 | W | 0.51 | 98.96 | 0.53 | 23.00 | 73.07 | 3.93 |
| | 2 | W | 0.48 | 97.75 | 1.78 | 29.06 | 65.15 | 5.79 |
| | 3 | W | 0.69 | 96.86 | 2.45 | 34.95 | 60.46 | 4.59 |
| His6.0 | 0 | d | 0.57 | 99.43 | — | 18.80 | 78.00 | 3.20 |
| | 1 | W | 0.50 | 99.01 | 0.50 | 22.98 | 73.57 | 3.44 |
| | 2 | W | 0.43 | 97.85 | 1.72 | 29.38 | 66.12 | 4.51 |
| | 3 | W | 0.66 | 96.92 | 2.41 | 35.33 | 60.93 | 3.74 |
| His6.5 | 0 | d | 0.56 | 99.44 | — | 18.91 | 78.01 | 3.07 |
| | 1 | W | 0.56 | 98.89 | 0.55 | 26.03 | 71.16 | 2.81 |
| | 2 | W | 0.42 | 97.82 | 1.77 | 36.09 | 60.06 | 3.25 |
| | 3 | W | 0.81 | 96.53 | 2.66 | 43.49 | 54.22 | 2.29 |

As shown by the SEC-HPLC monomer purity data in Table 2, His 6.0 and His 5.5 had smaller decline in SEC monomer purities, and were superior to His 6.5, NaAc 5.5, NaAc 5.0 and NaAc 6.0, and the monomer in His buffer had a good stability when pH ranged from 5.5 to 6.0. It can be seen from the main peak content data of the IEC-HPLC that His 6.0, His 5.5 and NaAc 6.0 were superior to NaAc 5.5, NaAc 5.0 and His 6.5, indicating that the IEC main peak content in the formulations had a good stability in a buffer with pH ranging from 5.5 to 6.0.

Based on the results of SEC-HPLC and IEC-HPLC, BAT4406F had a good stability at a high temperature (40′C) when pH of the antibody sample ranges from 5.5 to 6.0.

Screening of Buffer System

The antibody had a good stability under pH ranging from 5.5 to 6.0. Therefore, buffers with buffer capacity ranging from pH 5.5 to pH 6.0 were selected for screening. Five different buffers were prepared: succinic acid-sodium succinate buffer (Sua), citric acid-sodium citrate buffer (CB), phosphate buffer (PB), histidine-histidine hydrochloride buffer (His), and acetic acid-sodium acetate buffer (NaAc) (see Table 3). Purified BAT4406F was ultra-filtered and exchanged into the above five buffers, and the concentration of the antibody was adjusted to 20 mg/mL. Samples of the resulting formulations were subjected to a high temperature test at 40° C., analyzed on day 0, week 1, week 2 and week 3 by SEC-HPLC and IEC-HPLC respectively. The results are shown in Table 4.

TABLE 3
Buffer composition

| Buffer ingredient | pH | Abbreviation |
|---|---|---|
| PB | 5.7 | PB |
| Citric acid/sodium citrate | 5.7 | CB |
| Succinic acid/sodium succinate | 5.7 | Sua |
| Acetic acid/sodium acetate | 5.7 | NaAc |
| L-histidine/histidine hydrochloride | 5.7 | His |

TABLE 4
Changes in SEC and IEC of antibody in different buffers over time at 40° C.

| Testing item | | | SEC-HPLC | | | IEC-HPLC | | |
|---|---|---|---|---|---|---|---|---|
| Sample | Sampling | | | Monomer | | Acidic | Main | Basic |
| name | day | | Polymer | purity | Fragment | region | peak | region |
| PB | 0 | d | 0.28 | 99.72 | — | 37.61 | 50.73 | 11.66 |
| | 1 | W | 0.67 | 99.15 | 0.17 | 41.04 | 47.75 | 11.21 |
| | 2 | W | 0.88 | 98.82 | 0.31 | 46.18 | 43.86 | 9.97 |
| | 3 | W | 0.91 | 96.43 | 2.66 | 52.34 | 39.40 | 8.26 |
| CB | 0 | d | 0.31 | 99.69 | — | 37.54 | 50.63 | 11.84 |
| | 1 | W | 0.58 | 99.28 | 0.14 | 41.48 | 47.16 | 11.36 |
| | 2 | W | 0.73 | 99.02 | 0.24 | 46.40 | 43.40 | 10.20 |
| | 3 | W | 0.74 | 96.91 | 2.36 | 52.62 | 38.93 | 8.45 |
| Sua | 0 | d | 0.29 | 99.71 | — | 37.65 | 50.69 | 11.66 |
| | 1 | W | 0.60 | 99.26 | 0.14 | 41.18 | 47.30 | 11.52 |
| | 2 | W | 0.71 | 99.05 | 0.24 | 45.00 | 44.57 | 10.43 |
| | 3 | W | 0.78 | 96.83 | 2.39 | 52.55 | 39.03 | 8.42 |
| NaAc | 0 | d | 0.30 | 99.70 | — | 37.80 | 50.46 | 11.74 |
| | 1 | W | 0.54 | 99.32 | 0.15 | 40.66 | 47.91 | 11.43 |
| | 2 | W | 0.58 | 99.16 | 0.25 | 44.01 | 45.52 | 10.48 |
| | 3 | W | 0.62 | 96.89 | 2.49 | 49.51 | 41.62 | 8.87 |
| His | 0 | d | 0.26 | 99.74 | — | 37.51 | 50.86 | 11.63 |
| | 1 | W | 0.37 | 99.48 | 0.15 | 39.95 | 48.55 | 11.50 |
| | 2 | W | 0.40 | 99.34 | 0.26 | 43.05 | 46.92 | 10.02 |
| | 3 | W | 0.42 | 97.08 | 2.50 | 48.51 | 42.33 | 9.17 |

As shown in Table 4, His buffer was best to maintain the SEC-HPLC monomer purity under high temperature conditions. It can be seen from the IEC-HPLC main peak content data that His buffer and NaAc buffer were better than other buffers.

Based on the results of SEC-HPLC and IEC-HPLC, the antibody sample in His buffer had a better stability in SEC-HPLC and IEC-HPLC at a high temperature (40° C.) than those in other buffers.

Optimization of pH Value

(1) pH Value Optimization 1

The antibody samples had a good stability in histidine buffer with pH ranging from about 5.5 to about 6.0. Optimization of pH was performed. Purified BAT4406F was ultra-filtered and the solvent was exchanged to prepare formulations having five pH values: histidine buffer (pH 5.6), histidine buffer (pH 5.8), histidine buffer (pH 6.0), histidine buffer (pH 6.2) and histidine buffer (pH 6.4). The concentrations of the antibody were all adjusted to be 20 mg/mL, and the samples were subjected to a high temperature test at 40° C., sampled on day 0, week 1, week 2 and week 3, and analyzed by SEC-HPLC and IEC-HPLC respectively. The results are shown in Table 5.

TABLE 5
Changes in SEC-HPLC and IEC-HPLC over time under different pH conditions

| | SEC-HPLC | | | IEC-HPLC | | |
|---|---|---|---|---|---|---|
| Testing item | | Monomer | | Acidic | Main | Basic |
| Sample name | Polymer | purity | Fragment | region | peak | region |
| His5.6-0 d | 0.25 | 99.75 | 0.00 | 39.40 | 50.44 | 10.16 |
| His5.8-0 d | 0.24 | 99.76 | 0.00 | 38.02 | 51.79 | 10.19 |
| His6.0-0 d | 0.24 | 99.76 | 0.00 | 38.24 | 51.59 | 10.17 |
| His6.2-0 d | 0.24 | 99.76 | 0.00 | 38.41 | 51.50 | 10.09 |
| His6.4-0 d | 0.24 | 99.76 | 0.00 | 38.54 | 51.39 | 10.07 |
| His5.6-1 W | 0.25 | 99.51 | 0.24 | 44.35 | 48.63 | 7.03 |
| His5.8-1 W | 0.39 | 99.42 | 0.19 | 44.48 | 48.83 | 6.69 |
| His6.0-1 W | 0.30 | 99.51 | 0.19 | 45.29 | 48.57 | 6.13 |
| His6.2-1 W | 0.43 | 99.37 | 0.21 | 46.11 | 48.12 | 5.78 |
| His6.4-1 W | 0.53 | 99.25 | 0.21 | 47.11 | 47.51 | 5.38 |
| His5.6-2 W | 1.04 | 97.84 | 1.12 | 46.21 | 45.39 | 8.41 |
| His5.8-2 W | 0.65 | 99.01 | 0.34 | 46.67 | 45.33 | 8.01 |
| His6.0-2 W | 0.59 | 99.07 | 0.34 | 47.31 | 44.99 | 7.70 |
| His6.2-2 W | 0.62 | 99.04 | 0.34 | 48.64 | 44.08 | 7.27 |
| His6.4-2 W | 0.67 | 98.98 | 0.35 | 50.34 | 42.81 | 6.85 |
| His5.6-3 W | 0.79 | 97.46 | 1.75 | 50.44 | 43.03 | 6.53 |
| His5.8-3 W | 0.94 | 97.38 | 1.68 | 51.10 | 42.92 | 5.99 |
| His6.0-3 W | 1.06 | 97.35 | 1.59 | 52.48 | 41.85 | 5.66 |
| His6.2-3 W | 1.10 | 97.14 | 1.76 | 54.19 | 40.69 | 5.12 |
| His6.4-3 W | 1.01 | 97.26 | 1.73 | 55.96 | 39.41 | 4.63 |

As shown in Table 5, 1) the formulations had similar SEC-HPLC monomer purity under high temperature; 2) when pH ranged from 5.6 to 6.2, the IEC-HPLC main peaks of the samples decreased slowly and had a good stability.

Based on the results of SEC-HPLC and IEC-HPLC, the antibody sample had a good stability when the pH value of the buffer ranged from 5.6 to 6.2.

(2) pH Value Optimization 2

It could be seen from the pH value optimization 1 that the antibody sample had a good stability when pH ranged from 5.6 to 6.2, and the IEC-HPLC performance was poor when pH exceeded 6.2, but the stability of the sample could not be determined when pH was lower than 5.6, so that a pH screening test was performed again. Since the pH of His buffer was greatly affected by temperature, citric acid-PB(C—P) was used as a buffer this time, and six samples of BAT4406F with different pH values including pH 5.0, pH 5.3, pH 5.6, pH 5.9, pH 6.2 and pH 6.5 were obtained after ultra-filtration and solvent exchange. The antibody samples were subjected to a high temperature test at 40° C., sampled on day 0, week 1, week 2 and week 3, and analyzed by SEC-HPLC and IEC-HPLC respectively. The results are shown in Table 6.

TABLE 6
SEC-HPLC and IEC-HPLC under different pH conditions

| | SEC-HPLC | | | IEC-HPLC | | |
|---|---|---|---|---|---|---|
| Testing item | | Monomer | | Acidic | Main | Basic |
| Sample name | Polymer | purity | Fragment | region | peak | region |
| PH5.0-0 d | 0.97 | 98.89 | 0.13 | 27.99 | 64.42 | 7.59 |
| PH5.3-0 d | 0.98 | 98.89 | 0.13 | 28.41 | 64.08 | 7.51 |
| PH5.6-0 d | 0.96 | 98.92 | 0.12 | 28.50 | 64.14 | 7.36 |
| PH5.9-0 d | 0.99 | 98.89 | 0.13 | 28.80 | 63.82 | 7.37 |
| PH6.2-0 d | 1.00 | 98.88 | 0.12 | 28.90 | 63.84 | 7.26 |
| PH6.5-0 d | 1.03 | 98.85 | 0.12 | 28.89 | 63.93 | 7.18 |
| PH5.0-1 W | 0.89 | 98.85 | 0.26 | 33.20 | 56.98 | 9.82 |
| PH5.3-1 W | 0.96 | 98.82 | 0.22 | 33.30 | 57.95 | 8.75 |
| PH5.6-1 W | 1.01 | 98.79 | 0.20 | 32.81 | 59.13 | 8.06 |
| PH5.9-1 W | 1.10 | 98.72 | 0.18 | 32.39 | 59.92 | 7.69 |
| PH6.2-1 W | 1.13 | 98.70 | 0.17 | 32.46 | 60.08 | 7.46 |
| PH6.5-1 W | 1.24 | 98.56 | 0.19 | 33.15 | 59.67 | 7.18 |
| PH5.0-2 W | 1.09 | 98.41 | 0.50 | 41.97 | 49.93 | 8.10 |
| PH5.3-2 W | 1.06 | 98.54 | 0.39 | 41.75 | 50.81 | 7.44 |
| PH5.6-2 W | 1.05 | 98.63 | 0.32 | 40.57 | 52.67 | 6.76 |
| PH5.9-2 W | 1.16 | 98.54 | 0.30 | 39.71 | 53.92 | 6.38 |
| PH6.2-2 W | 1.20 | 98.51 | 0.29 | 39.41 | 54.56 | 6.03 |
| PH6.5-2 W | 1.34 | 98.34 | 0.32 | 40.42 | 53.91 | 5.67 |
| PH5.0-3 W | 1.01 | 95.94 | 3.06 | 47.06 | 43.61 | 9.33 |
| PH5.3-3 W | 1.00 | 96.41 | 2.60 | 46.95 | 44.65 | 8.40 |
| PH5.6-3 W | 1.16 | 96.48 | 2.36 | 45.71 | 46.51 | 7.78 |
| PH5.9-3 W | 1.19 | 96.55 | 2.26 | 44.17 | 48.53 | 7.30 |
| PH6.2-3 W | 1.28 | 96.43 | 2.29 | 43.53 | 48.64 | 7.84 |
| PH6.5-3 W | 1.35 | 96.36 | 2.30 | 45.41 | 48.17 | 6.41 |
| PH5.0-4 W | 1.12 | 95.23 | 3.66 | 52.73 | 39.69 | 7.59 |
| PH5.3-4 W | 1.16 | 95.79 | 3.05 | 52.80 | 40.44 | 6.76 |
| PH5.6-4 W | 1.21 | 96.11 | 2.69 | 51.43 | 42.43 | 6.14 |
| PH5.9-4 W | 1.26 | 96.24 | 2.50 | 49.24 | 45.11 | 5.65 |
| PH6.2-4 W | 1.30 | 96.24 | 2.46 | 48.83 | 45.97 | 5.19 |
| PH6.5-4 W | 1.38 | 96.14 | 2.48 | 50.47 | 44.70 | 4.84 |

As shown in Table 6, 1) the SEC-HPLC monomer purities of the samples decreased after being placed under high temperature for four weeks, and the monomer purities were poor when pH<5.3; 2) when pH ranged from 5.6 to 6.5, IEC-HPLC main peaks of the samples decreased slowly and had a good stability.

pH value is a key factor for the liquid formulations. The antibody showed different stabilities in buffers with different pH values, but when the pH value fluctuated within a certain range, the quality of the antibody would not be affected significantly. Based on the above studies of primary screening of pH and fine screening of pH, it was preliminarily determined that the antibody had a good stability in the pH range from 5.5 to 6.7.

Screening of Stabilizers

Solutions of BAT4406F (50 mg/ml) containing seven stabilizers, sodium chloride (0.9%), sucrose (9%), trehalose (9%), mannitol (4%), sorbitol (4%), arginine (1%) and methionine (1%), were prepared respectively, and the specific formulations are shown in Table 7. Tre (trehalose) in Table 7 referred to trehalose dihydrate.

TABLE 7
Formulations of different stabilizers

| Serial number | Formulation code | Ingredient |
|---|---|---|
| 1 | Blank | Antibody |
| 2 | NaCl | Antibody + sodium chloride |
| 3 | Suc | Antibody + sucrose |
| 4 | Tre | Antibody + trehalose |
| 5 | Man | Antibody + mannitol |
| 6 | Sor | Antibody + sorbitol |
| 7 | Arg | Antibody + arginine |
| 8 | Met | Antibody + methionine |

(1) High Temperature Test

The samples with stabilizers were compared with a sample without stabilizers at a high temperature (40 V), and the changes in the quality of the samples were investigated. The samples were analyzed at week 1, week 2, week 3 and week 4 by SEC-HPLC. The results are shown in Table 8.

TABLE 8
Changes in SEC-HPLC of samples with
different stabilizers over time

| Testing item | SEC-HPLC | | |
|---|---|---|---|
| Sample name | Polymer | Monomer purity | Fragment |
| Blank-0 d | 0.28 | 99.74 | — |
| NaCl-0 d | 0.33 | 99.67 | — |
| Suc-0 d | 0.22 | 99.78 | — |
| Tre-0 d | 0.24 | 99.76 | — |
| Man-0 d | 0.21 | 99.79 | — |
| Sor-0 d | 0.22 | 99.78 | — |
| Arg-0 d | 0.60 | 99.41 | — |
| Met-0 d | 0.20 | 99.80 | — |
| Blank-1 W | 0.70 | 99.12 | 0.19 |
| NaCl-1 W | 0.93 | 98.91 | 0.16 |
| Suc-1 W | 0.55 | 99.27 | 0.17 |
| Tre-1 W | 0.52 | 99.32 | 0.16 |
| Man-1 W | 0.68 | 99.11 | 0.21 |
| Sor-1 W | 0.66 | 99.22 | 0.13 |
| Arg-1 W | 0.84 | 99.03 | 0.13 |
| Met-1 W | 0.78 | 98.83 | 0.38 |
| Blank-2 W | 0.67 | 97.42 | 0.33 |
| NaCl-2 W | 1.13 | 96.90 | 1.97 |
| Suc-2 W | 0.54 | 97.49 | 1.97 |
| Tre-2 W | 0.47 | 97.67 | 1.87 |
| Man-2 W | 0.58 | 97.50 | 1.92 |
| Sor-2 W | 0.59 | 97.58 | 0.83 |
| Arg-2 W | 0.82 | 97.44 | 1.75 |
| Met-2 W | 0.54 | 97.48 | 1.98 |
| Blank-3 W | 0.90 | 97.29 | 1.81 |
| NaCl-3 W | 1.25 | 97.07 | 1.67 |
| Suc-3 W | 0.82 | 97.17 | 2.01 |
| Tre-3 W | 0.75 | 97.51 | 1.75 |
| Man-3 W | 0.96 | 97.05 | 2.00 |
| Sor-3 W | 0.71 | 97.28 | 2.00 |
| Arg-3 W | 0.90 | 97.15 | 1.95 |
| Met-3 W | 0.70 | 97.17 | 2.13 |
| Blank-4 W | 1.11 | 96.20 | 2.69 |
| NaCl-4 W | 1.57 | 95.99 | 2.44 |
| Suc-4 W | 0.76 | 96.84 | 2.40 |
| Tre-4 W | 0.73 | 97.05 | 2.22 |
| Man-4 W | 0.93 | 96.50 | 2.57 |
| Sor-4 W | 0.74 | 97.00 | 2.25 |
| Arg-4 W | 1.05 | 96.47 | 2.48 |
| Met-4 W | 0.72 | 96.50 | 2.78 |

As shown in Table 8, Tre, Sor and Suc showed the best SEC-HPLC monomer purity.

(2) Light Condition Test

The samples with stabilizers were compared under light to investigate the changes in the quality of the samples. The samples were analyzed at week 1 and week 2 by SEC-HPLC and IEC-HPLC detections. The results are shown in Table 9.

TABLE 9
Changes in SEC-HPLC and IEC-HPLC of samples with different stabilizers
over time (25° C., 4000 ± 5001x)

| | SEC-HPLC | | | IEC-HPLC | | |
|---|---|---|---|---|---|---|
| Testing item | | Monomer | | Acidic | Main | Basic |
| Sample name | Polymer | purity | Fragment | region | peak | region |
| Blank-0 d | 0.28 | 99.74 | — | 38.27 | 50.99 | 10.74 |
| NaCl-0 d | 0.33 | 99.67 | — | 38.21 | 51.09 | 10.70 |
| Suc-0 d | 0.22 | 99.78 | — | 38.36 | 51.03 | 10.61 |
| Tre-0 d | 0.24 | 99.76 | — | 38.37 | 51.04 | 10.59 |
| Man-0 d | 0.21 | 99.79 | — | 38.35 | 51.09 | 10.56 |
| Sor-0 d | 0.22 | 99.78 | — | 38.38 | 51.06 | 10.56 |
| Arg-0 d | 0.60 | 99.41 | — | 38.36 | 51.08 | 10.56 |
| Met-0 d | 0.20 | 99.80 | — | 38.44 | 51.10 | 10.46 |
| Blank-1 W | 3.51 | 96.23 | 0.27 | 67.08 | 31.87 | 1.05 |
| NaCl-1 W | 3.73 | 94.43 | 1.84 | 62.35 | 36.11 | 1.53 |
| Suc-1 W | 1.93 | 97.83 | 0.23 | 63.01 | 36.09 | 0.89 |
| Tre-1 W | 1.83 | 97.95 | 0.22 | 62.86 | 36.18 | 0.96 |
| Man-1 W | 2.49 | 97.27 | 0.24 | 64.83 | 34.44 | 0.73 |
| Sor-1 W | 2.04 | 97.73 | 0.23 | 63.81 | 35.35 | 0.84 |
| Arg-1 W | 2.33 | 95.05 | 2.61 | 60.82 | 38.16 | 1.02 |
| Met-1 W | 1.11 | 98.89 | 0.00 | 57.44 | 41.50 | 1.06 |
| Blank-2 W | 7.03 | 92.51 | 0.47 | 59.76 | 30.46 | 9.78 |
| NaCl-2 W | 8.15 | 91.32 | 0.53 | 53.16 | 34.34 | 12.49 |
| Suc-2 W | 3.45 | 96.07 | 0.48 | 54.95 | 36.86 | 8.19 |
| Tre-2 W | 3.48 | 96.07 | 0.45 | 53.70 | 37.61 | 8.70 |
| Man-2 W | 4.16 | 95.35 | 0.49 | 55.42 | 35.74 | 8.84 |
| Sor-2 W | 3.92 | 95.61 | 0.47 | 54.79 | 36.33 | 8.89 |
| Arg-2 W | 4.53 | 95.01 | 0.46 | 48.24 | 40.71 | 11.05 |
| Met-2 W | 1.54 | 98.10 | 0.36 | 46.47 | 44.60 | 8.93 |

As shown in Table 9, 1) the SEC-HPLC monomer purity of the sample Met declined the least, followed by the samples Tre, Suc and the other; 2) the IEC-HPLC main peak content of Met and Arg were good, followed by Tre, Sue and Sor, etc.

Based on the results of high temperature and light condition, trehalose performed well in both SEC-HPLC and IEC-HPLC.

Screening of Surfactants

Compatibility Test with Normal Saline

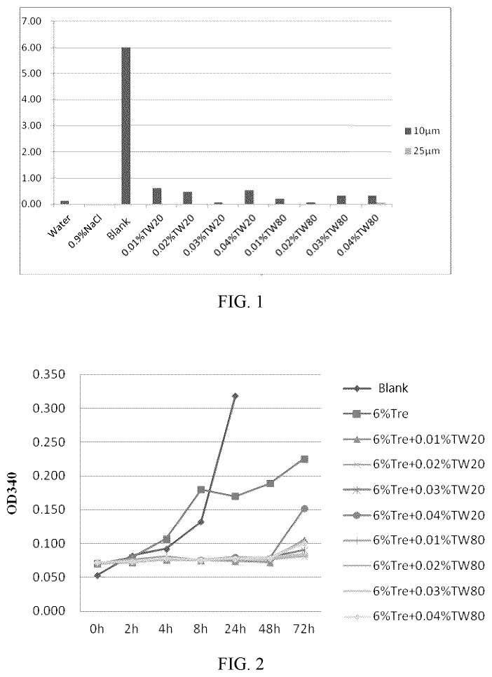

When being administered by intravenous injection, an antibody formulation needs to be formulated with normal saline clinically, so a compatibility test was performed. Antibody sample solutions with surfactants of different concentrations and different types were prepared, and the specific formulations are shown in Table 10. The concentration of BAT4406F in the sample solutions was 30 mg/mL, the sample solutions containing surfactants of different concentrations in the same volume were respectively added into 0.9% sodium chloride solution according to a compatibility ratio (1 mL sample:29 mL normal saline), characters thereof were observed after slight and full mixing, and insoluble particle analysis was performed on the antibody solutions after standing for 2 hours. The results are shown in FIG. 1.

TABLE 10
Formulations of antibody solutions

| Formulation Name | Ingredient |
|---|---|
| Blank | Antibody (without surfactant) |
| 0.01% TW20 | 0.01% Tween 20 + antibody |
| 0.02% TW20 | 0.02% Tween 20 + antibody |
| 0.03% TW20 | 0.03% Tween 20 + antibody |
| 0.04% TW20 | 0.04% Tween 20 + antibody |
| 0.01% TW80 | 0.01% Tween 80 + antibody |
| 0.02% TW80 | 0.02% Tween 80 + antibody |
| 0.03% TW80 | 0.03% Tween 80 + antibody |
| 0.04% TW80 | 0.04% Tween 80 + antibody |

As shown in FIG. 1, the blank without surfactants had the largest number of particles, while those with surfactants (polysorbate-80 (Tween 80) or polysorbate-20 (Tween 20)) generally had smaller numbers of particles and the number of particles was slightly reduced when the surfactant content was higher: however, when the concentration of the surfactant reached a certain level, the number of particles in the sample increased again. When the surfactant was polysorbate-80 with a concentration of 0.02%, the number of particles in the formulation was small.

Shaking Test

Shaking was inevitable when transporting or transferring samples, which can accelerate protein aggregation. Adding a surfactant in a formulation may effectively prevent the protein aggregation. Sample solutions of BAT4406F (20 mg/ml) containing surfactants of different concentrations and different types were prepared, and the specific formulations are shown in Table 11. The samples were placed flat on a shaker, and the antibody solutions were subjected to the shaking test at 200 rpm at room temperature. Light inspection and turbidity (OD340 value) analysis were performed on the solutions at 0 h, 2 h, 4 h, 8 h, 24 h, 48 h and 72 h to investigate the changes in the quality of the antibody solutions. Results are shown in FIG. 2 (note: the OD value was the numerical value obtained by detecting the protein solution at UV340 nm), Tre in Table 11 referred to trehalose dihydrate.

TABLE 11
Formulations of antibody solutions

| Formulation Name | Ingredient |
|---|---|
| Blank | Antibody (without surfactants and stabilizers) |
| 6% Tre | 6% Tre + antibody |
| 6% Tre + 0.01% TW20 | 6% Tre + 0.01% TWEEN20 + antibody |
| 6% Tre + 0.02% TW20 | 6% Tre + 0.02% TWEEN20 + antibody |
| 6% Tre + 0.03% TW20 | 6% Tre + 0.03% TWEEN20 + antibody |
| 6% Tre + 0.04% TW20 | 6% Tre + 0.04% TWEEN20 + antibody |
| 6% Tre + 0.01% TW80 | 6% Tre + 0.01% TWEEN80 + antibody |
| 6% Tre + 0.02% TW80 | 6% Tre + 0.02% TWEEN80 + antibody |
| 6% Tre + 0.03% TW80 | 6% Tre + 0.03% TWEEN80 + antibody |
| 6% Tre + 0.04% TW80 | 6% Tre + 0.04% TWEEN80 + antibody |

As shown in FIG. 2, the blank sample and the sample added with only 6% Tre started to be turbid after being shaking for 4 hours, and the sample having Tween remained clear after shaking for 72 hours, indicating that polysorbate-80 or polysorbate-20 had a certain protective effect on the samples under shaking and could prevent protein aggregation and becoming turbid, and played a role of solubilization; for both polysorbate-80 and polysorbate-20, the 0.02% and 0.03% samples had the lowest turbidity, and the 0.01% and 0.04% samples had a relatively high turbidity, indicating that the content of polysorbate should be neither too low nor too high. 0.02% polysorbate was preferred.

Repeated Freezing and Thawing Test 1

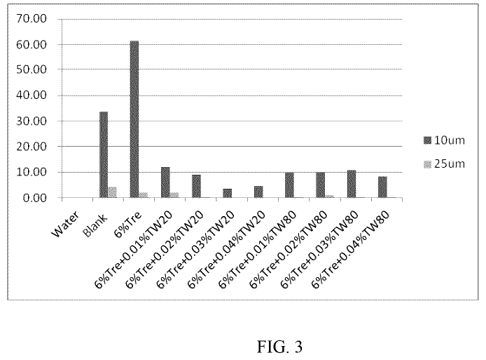

Samples of BAT4406F (20 mg/ml) with different polysorbate (i.e., Tween) contents and types were prepared for repeated freezing and thawing test (the specific formulations are shown in Table 12, and Tre in Table 12 referred to trehalose dihydrate). After being frozen at −80° C. for 24 hours, the sample solutions were placed at 25° C. for 24 hours for one cycle of freezing and thawing, which was performed 5 times. Insoluble particle analysis was performed on the frozen and thawed samples, and results are shown in FIG. 3.

TABLE 12
Formulations of antibody solutions

| Formulation number | Ingredient |
|---|---|
| Blank | Antibody (without surfactants and stabilizers) |
| 6% Tre | 6% Tre + antibody |
| 6% Tre + 0.01% TW20 | 6% Tre + 0.01% TWEEN20 + antibody |
| 6% Tre + 0.02% TW20 | 6% Tre + 0.02% TWEEN20 + antibody |
| 6% Tre + 0.03% TW20 | 6% Tre + 0.03% TWEEN20 + antibody |
| 6% Tre + 0.04% TW20 | 6% Tre + 0.04% TWEEN20 + antibody |
| 6% Tre + 0.01% TW80 | 6% Tre + 0.01% TWEEN80 + antibody |
| 6% Tre + 0.02% TW80 | 6% Tre + 0.02% TWEEN80 + antibody |
| 6% Tre + 0.03% TW80 | 6% Tre + 0.03% TWEEN80 + antibody |
| 6% Tre + 0.04% TW80 | 6% Tre + 0.04% TWEEN80 + antibody |

As shown in FIG. 3, after repeated freezing and thawing for five times, the number of particles in the sample with Tween was obviously smaller than that in the samples without Tween, indicating that the Tween could effectively prevent protein aggregation during repeated freezing and thawing.

### Example 2

A liquid formulation comprising BAT4406F was provided herein, consisting of the following ingredients: about 15 mg/ml BAT4406F, about 10 mM succinate buffer, about 80 mM sucrose and about 0.1 mg/ml polysorbate-20, and the pH of the liquid formulation was about 5.5.

### Example 3

A liquid formulation comprising BAT4406F was provided herein, consisting of the following ingredients: about 40 mg/ml anti-CD20 antibody, about 15 mM citrate buffer, about 140 mM sorbitol and about 0.3 mg/ml polysorbate-80, and the pH value of the liquid formulation was about 5.6.

### Example 4

A liquid formulation comprising BAT4406F was provided herein, consisting of the following ingredients: about 60 mg/ml anti-CD20 antibody, about 26 mM acetate buffer, about 190 mM sorbitol and about 0.35 mg/ml polysorbate-20, and the pH value of the liquid formulation was about 5.7.

### Example 5

A liquid formulation comprising BAT4406F was provided herein, consisting of the following ingredients: about 70 mg/ml anti-CD20 antibody, about 26 mM phosphate buffer, about 240 mM methionine and about 0.38 mg/ml polysorbate-20, and the pH value of the liquid formulation was about 6.0.

### Example 6

A liquid formulation comprising BAT4406F was provided herein, consisting of the following ingredients: about 80 mg/ml anti-CD20 antibody, about 30 mM phosphate buffer, about 158 mM mannitol and about 0.4 mg/ml polysorbate-20 and the pH value of the liquid formulation was about 6.2.

### Example 7

A liquid formulation comprising BAT4406F was provided herein, consisting of the following ingredients: about 20 mg/ml anti-CD20 antibody, about 20 mM histidine buffer, about 158.6 mM trehalose and about 0.2 mg/ml polysorbate-80, and the pH value of the liquid formulation was about 5.8. Based on the pH value of 5.8, the molar ratio of L-histidine to L-histidine hydrochloride in the histidine buffer was 2:3.

### Example 8

A liquid formulation comprising BAT4406F was provided herein, consisting of the following ingredients: about 20 mg/nil anti-CD20 antibody, about 20 mM histidine buffer, about 224.6 mM trehalose and about 0.2 mg/ml polysorbate-80, and the pH value of the liquid formulation was about 5.8. Based on the pH value of 5.8, the molar ratio of L-histidine to L-histidine hydrochloride in the histidine buffer was 2:3.

### Example 9

A liquid formulation comprising BAT4406F was provided herein, consisting of the following ingredients: about 50 mg/ml anti-CD20 antibody, about 20 mM histidine buffer, about 224.6 mM trehalose and about 0.2 mg/ml polysorbate-80, and the pH value of the liquid formulation was about 5.8. Based on the pH value of 5.8, the molar ratio of L-histidine to L-histidine hydrochloride in the histidine buffer was 2:3.

### Example 10

A liquid formulation comprising BAT4406F was provided herein, consisting of the following ingredients: about 30 mg/ml anti-CD20 antibody, about 18 mM histidine buffer, about 170 mM trehalose and about 0.18 mg/ml polysorbate-80, and the pH value of the liquid formulation was about 5.7.

### Example 11

A liquid formulation comprising BAT4406F was provided herein, consisting of the following ingredients: about 40 mg/ml anti-CD20 antibody, about 19 mM histidine buffer, about 225 mM trehalose and about 0.19 mg/ml polysorbate-80, and the pH value of the liquid formulation was about 5.9.

### Example 12

A liquid formulation comprising BAT4406F was provided herein, consisting of the following ingredients: about 46 mg/ml anti-CD20 antibody, about 22 mM histidine buffer, about 220 mM trehalose and about 0.22 mg/ml polysorbate-80, and the pH value of the liquid formulation was about 6.0.

Stability Comparison

The Formulation (with an antibody concentration of 20 mg/mL) disclosed in Chinese Patent CN101820912B (Formulation A, the antibody is ofatumumab), a comparative Formulation (using the same excipients as those in the formulation of CN101820912B, but the antibody thereof was BAT4406F, Formulation B), Formulation 1 (see Example 7), Formulation 2 (Example 8), Formulation 3 (Example 9) and Formulation 4 were used for stability analysis. The formulations are shown in Table 13.

TABLE 13
Prescriptions of formulations

| | Formulation | | | | | |
|---|---|---|---|---|---|---|
| | Concentration | | | | | |
| Name | of antibody | | Buffer system | Stabilizer | Surfactant | pH |
| Formulation A | 20 | mg/mL | 50 mM sodium | 1% arginine + 51 mM sodium | 0.02% TWEEN80 | 5.5 |
| | | | acetate | chloride + 0.05 mM EDTA | | |
| Formulation B | 20 | mg/mL | 50 mM sodium | 1% arginine + 51 mM sodium | 0.02% TWEEN80 | 5.5 |
| | | | acetate | chloride + 0.05 mM EDTA | | |
| Formulation 1 | 20 | mg/mL | 20 mM histidine | 158.6 mM trehalose | 0.02% TWEEN80 | 5.8 |
| Formulation 2 | 20 | mg/mL | 20 mM histidine | 224.6 mM trehalose | 0.02% TWEEN80 | 5.8 |
| Formulation 3 | 50 | mg/mL | 20 mM histidine | 224.6 mM trehalose | 0.02% TWEEN80 | 5.8 |
| Formulation 4 | 100 | mg/mL | 20 mM histidine | 224.6 mM trehalose | 0.02% TWEEN80 | 5.8 |

1) Dissolution Temperature, Aggregation Temperature and Particle Size

TABLE 14
Dissolution temperature, aggregation temperature and particle size

| | Concentration | | Uncle test data | | | |
|---|---|---|---|---|---|---|
| Name | of antibody | | Tm1 | Tagg | PDI | PK1 Mode Dia |
| Formulation A | 20 | mg/mL | 65.09 | 63.57 | 0.10 | 11.52 |
| Formulation B | 20 | mg/mL | 64.73 | 63.53 | 0.13 | 11.52 |
| Formulation 1 | 20 | mg/mL | 70.50 | 68.68 | 0.23 | 8.45 |
| Formulation 2 | 20 | mg/mL | 71.03 | 69.11 | 0.64 | 9.87 |
| Formulation 3 | 50 | mg/mL | 71.00 | 68.40 | 1.98 | 8.45 |
| Formulation 4 | 100 | mg/mL | 69.68 | 66.80 | 3.68 | 7.23 |

As shown in Table 14, 1) when the anti-CD20 antibody in the original Formulation A was replaced by SAT4406F, Tm1, Tagg and DLS (particle size and uniformity) of the two formulations were basically the same; 2) compared with the original Formulation A, Tm1 and Tagg of the formulation of the Formulation 1 were both high, and the PDI of the particle size was less than 0.25, and the uniformity of the formulation was good, indicating that the Formulation 1 had a good stability; 3) when trehalose was adjusted from 158.6 mM to 224.6 mM and the others remained unchanged, Tm1, Tagg and DLS of Formulation 2 and Formulation 3 were basically consistent with those of Formulation 1, and the stability was relatively good.

2) High Temperature Test at 49° C.

Samples of the formulations were subjected to a high temperature test at 40° C., analyzed on day 0, week 1, week 2, week 3 and week 4 by SEC-HPLC. The results are shown in Table 15,

As shown by the SEC-HPLC monomer purity data in Table 15, Formulation 1, Formulation 2 and Formulation 3 had a better stability compared with the Formulation B, with Formulation 1 and Formulation 2 having the highest stability.

TABLE 15
Monomer purity of the formulations under 40° C. by SEC

| | Concentration | | | | | | |
|---|---|---|---|---|---|---|---|
| Formulation | of antibody | | 0 d | 1 W | 2 W | 3 W | 4 W |
| Formulation B | 20 | mg/mL | 98.48 | 98.43 | 97.96 | 97.83 | 95.06 |
| Formulation 1 | 20 | mg/mL | 98.55 | 98.7 | 98.34 | 98.5 | 95.97 |
| Formulation 2 | 20 | mg/mL | 98.58 | 98.76 | 98.51 | 98.54 | 95.33 |
| Formulation 3 | 50 | mg/mL | 98.58 | 98.59 | 98.3 | 98.21 | 94.95 |
| Formulation 4 | 100 | mg/mL | 98.49 | 98.18 | 97.83 | 97.67 | 92.38 |

3) Light Condition Test

The formulations were analyzed for monomer purity on day 0, day 5 and day 10 by SEC-HPLC after being exposed to light. The results are shown in Table 16.

As shown in Table 16, The SEC-HPLC monomer purity of the formulations of the formulation 1, the formulation 2 and the formulation 3 had a better stability; especially, compared with the Formulation B, the Formulation 1 and the Formulation 2 had a better stability.

TABLE 16
Changes in monomer purity of formulations over
time (25° C., 4000 ± 5001x)

| GZ-SEC | 0 d | 5 d | 10 d |
|---|---|---|---|
| Formulation B-20 mg/mL | 98.48 | 97.15 | 95.03 |
| Formulation 1-20 mg/mL | 98.55 | 98.44 | 97.42 |
| Formulation 2-20 mg/mL | 98.58 | 98.4 | 97.82 |
| Formulation 3-50 mg/mL | 98.58 | 97.42 | 95.41 |
| Formulation 4-100 mg/mL | 98.49 | 94.9 | 90.33 |

Compatibility Analysis

The liquid formulations of the present invention had a good compatibility with the drug vials and met the packaging requirements through an accelerated stability test and a long-term stability test.

### Example 13. Clinical Study of BAT4406F

This study is a phase I, open-label and dose escalation clinical study evaluating the safety, tolerability, pharmacokinetics and preliminary effectiveness of BAT4406F in the treatment of patients with neuromyelitis optica spectrum disorders (NMOSD).

Subject Enrollment/Screening

Inclusion criteria include:
1) Compliance with the NMOSD diagnostic criteria established by the International Panel for NMO Diagnosis (IPND) in 2015 (see Appendix 1);
2) ≥18 and ≤65 years old;
3) At least two relapses occurred within two years before screening, or at least one relapse within one year before screening;
4) Immunosuppressive agents such as azathioprine, tacrolimus, mycophenolate mofetil, cyclophosphamide and methotrexate are discontinued within 28 days before the baseline;
5) The amount of corticosteroid is equivalent to 30 mg of prednisone or less, and must be stopped within one month after the administration of test drug; and
6) EDSS score ≤6.

Exclusion criteria include:
1) Treatment with any monoclonal antibody within six months prior to administration;
2) Having been treated with anti-CD20 mAb;
3) Having received live vaccine within four weeks before screening;
4) Having participated in another clinical study within 1 months or 5 half-lives of the drug prior to the baseline (whichever is longer);
5) A history of allergies to monoclonal antibodies; severe allergic reaction to certain foods or drugs;
6) Abnormal liver function, kidney function and bone marrow reserve:
a. ALT≥2.5×ULN and/or AST≥2.5×ULN and/or TBIL≥1.5×ULN:
b. serum creatinine ≥1.5×ULN, creatinine clearance 60 mL/min (calculated using the Cockcroft formula);
c. patients with a blood neutrophil count <1.5×10⁹/L and/or a platelet count <75×10⁹/L;
7) History of infections that investigators have identified as unsuitable for testing;
8) Patients with acute respiratory failure and pulmonary interstitial infiltration and edema seen in chest CT;
9) Patients with a clear history of heart disease (such as angina pectoris, atrial flutter and atrial fibrillation, conduction block and other arrhythmias or heart failure); for patients with degree I atrioventricular block, it is determined by the investigator in combination with the medical history; or
10) Patients also diagnosed with malignant tumors.

Test Drug, Usage and Dosage:

BAT4406F is intravenously administered at a dose of 20 mg, 50 mg, 100 mg, 200 mg, 500 mg. 1000 mg, or 2000 mg. Each administration is followed by an observation period of 6 months.

Prophylactic administration of an anti-histamine (such as diphenhydramine, promethazine hydrochloride, etc.) and glucocorticoids (such as methylprednisolone, dexamethasone, etc.) is conducted before the start of BAT4406F administration.

The initial intravenous infusion rate is about 12 ml/hour. If no infusion reaction occurs, the rate is increased by 12 ml/hour every half hour until the maximum infusion rate of 96 ml/hour.

Safety, tolerance, clinical pharmacokinetics (such as Cₘₐₓ, Tₘₐₓ, t_{1/2}, CLₜ, Vd, MRT, AUC_{(0-f)}, AUC_{(0-inf)}), pharmacodynamics, immunogenicity and preliminary effectiveness (evaluated by e.g., annual recurrence rate (ARR), first recurrence time, expanded disability status scale (EDSS), MRI, and the like) will be assessed. MRI may be evaluated according to International Consensus Diagnostic Criteria for Neuromyelitis Optica Spectrum Disorders (Neurology, 2015; 85:177-189). After receiving the treatment, the patient may achieve a complete response, a partial response, or stable disease. In some embodiments, the patient achieves a reduction in the ARR and EDSS score (e.g., the score reduces by at least 0.5).

Clinical Results

The two subjects enrolled in the 20 mg dose group experienced infusion reactions on the day of administration, which recovered/relieved by themselves without treatments, and the first subject has completed the DLT observation period, indicating that the current 20 mg dosage is well tolerated.

The proportion of CD19⁺B cells of both subjects decreased by over 80% to over 90% when tested on Day 4, Day 8 and Day 15 after administration of 20 mg of BAT4406F.

### Example 14. Non-Clinical Study of BAT4406F

1. Pharmacodynamic Study

The pharmacodynamic study of B cell depletion in cynomolgus monkeys showed that the onset time of BAT4406F was not significantly related to the dose in the tested dose range (2, 5, 15 mg/kg). On day 2 after administration, the CD20⁺ lymphocyte subgroups were below 0.5% in all three dose groups, and the ratio was maintained until day 21. On day 28 after administration, the proportion of the CD20⁺ lymphocyte subgroup in the low, medium, and high dose groups were restored to 3.87%, 2.43%, and 0.3%, respectively. At the same dose of 5 mg/kg, BAT4406F showed better B cell depletion ability than the marketed drug Rituximab.

The in vitro pharmacodynamic study using healthy human whole blood demonstrated that both 10 ng/mL and 50 ng/mL of BAT4406F showed strong ability to deplete B cells after incubated with whole blood for 4 h. The average percentages of B-cell depletion at the above concentrations were 37.16% and 61.99%, respectively, and the efficacy was dose-related. Compared with the commercially available anti-CD20 monoclonal antibodies, the ability of B cell depletion from strong to weak was BAT4406F>ofatumumab>ocrelizumab>rituximab.

See Table 17 for details.

TABLE 17
Pharmacodynamic study results

| Experiment | Dosage | BAT4406F | Rituximab | Ofatumumab | Ocrelizumab |
|---|---|---|---|---|---|
| Percentage | 50 ng/ml | 61.99% | 35.96% | 53.16% | 48.26% |
| of whole | | (n = 12) | (n = 5) | (n = 5) | (n = 4) |
| blood B-cell | 10 ng/ml | 37.16% | 17.75% | 26.96% | 22.18% |
| depletion | | (n = 12) | (n = 5) | (n = 5) | (n = 4) |
| in vitro | | | | | |
| B-cell | 5 mg/kg | The percentages of | The percentages of | NA | NA |
| depletion in | intravenous | CD20⁺ lymphocytes | CD20⁺ lymphocytes | | |
| cynomolgus | infusion, | in the blood were: | in the blood were: | | |
| monkeys | single-dose | before administration | before administration | | |
| | | (32.04%), | (31.04%), | | |
| | | D 2 (0.06%), | D 2 (0.09%), | | |
| | | D 7 (0.05%), | D 7 (0.05%), | | |
| | | D 14 (0.03%), | D 14 (1.08%), | | |
| | | D 21 (0.47%), | D 21 (4.21%), | | |
| | | D 28 (2.43%); | D 28 (5.44%); | | |
| | | the percentages of | the percentages of | | |
| | | CD40⁺ lymphocytes | CD40⁺ lymphocytes | | |
| | | in the blood were: | in the blood were: | | |
| | | before administration | before administration | | |
| | | (12.62%), | (13.68%), | | |
| | | D 2 (1.43%), | D 2 (1.74%), | | |
| | | D 7 (0.56%), | D 7 (2.16%), | | |
| | | D 14 (0.32%), | D 14 (1.54%), | | |
| | | D 21 (0.52%), | D 21 (2.45%), | | |
| | | D 28 (1.79%). | D 28 (3.78%). | | |
| | | D 29 the percentages | D 29 the percentages | | |
| | | of CD20⁺ lymphocytes | of CD20⁺ lymphocytes | | |
| | | and CD40⁺ lymphocytes | and CD40⁺ lymphocytes | | |
| | | in spleen were | in spleen were | | |
| | | 20.72% and 0.35%, | 14.59% and 0.38%, | | |
| | | respectively. | respectively. | | |

“NA” indicates that no test was conducted.

2. Pharmacological Research

The target of BAT4406F is the CD20 antigen expressed by B cells. After binding to CD20 antigen, BAT4406F can initiate an immune response that mediates B cell lysis. The depleting mechanism may include: antibody-dependent cell-mediated cytotoxicity (ADCC); antibody-dependent complement-mediated cytotoxicity (CDC); directly inducing apoptosis of CD20⁺ B cells.

The ADCC activity was evaluated by the PBMC cell killing method and the reporter gene assay method. Different anti-CD20 monoclonal antibodies BAT4406F, BAT4406 (fucose-modified protein expressed by wild type cell CHO-BAT), ofatumumab, ocrelizumab and rituximab were able to induce ADCC effect and the ADCC effect of BAT4406F obtained after knocking out fucose was enhanced by about 10-fold, which was significantly stronger than the other four anti-CD20 monoclonal antibodies, indicating that the ADCC effect was one of the main mechanisms of action of BAT4406F.

See Table 18 for details.

TABLE 18
Summary of In Vitro Activities

| Research content | | BAT4406F | | BAT4406 | | Ofatumumab | | Ocrelizumab | | Rituximab | |
|---|---|---|---|---|---|---|---|---|---|---|---|
| Binding to CD 20⁺ cells | | 5.49 | nM | 4.76 | nM | 4.16 | nM | 6.35 | nM | 10.17 | nM |
| ADCC | PBMC method | 0.0028 | nM | 0.0241 | nM | 0.0261 | nM | 0.0215 | nM | 0.0496 | nM |
| | Reporter gene | 0.001 | nM | 0.012 | nM | 0.011 | nM | 0.010 | nM | 0.019 | nM |

| | | | | | | |
|---|---|---|---|---|---|---|
| | assay method | | | | | |
| Cell | Raji | 2.59% | NA | NA | NA | 3.69% |
| apoptosis | Wil2-s | 2.29% | | | | 4.97% |

“NA” indicates that this item was not detected.

3. Toxicology Study

Long-term toxicity study was performed by administering BAT4406F to cynomolgus monkeys at 10 mg/kg, 30 mg/kg and 100 mg/kg, once a week for 4 consecutive weeks, for a total of 5 times, and the recovery period was 20 weeks. No significant abnormal changes associated with drug administration were observed in body weight, body temperature, electrocardiogram, blood pressure, coagulation function, clinical biochemistry, serum complement C3 and C4, immunoglobulin IgG, IgM and IgA, ophthalmologic examination, bone marrow smear examination, organ weight, organ-to-body weight ratio and organ-to-brain weight ratio in any dose group. Blood cell count showed lymph (×10⁹/L) decrease in the female animals in each dose group, and Mono (%) increase in the male animals in the 30 mg/kg group the day after the third administration (D16); Mono (%) increase in male animals in the 10 mg/kg group, MCHC decrease in female and male animals, Retic (%) increase in female animals in the 30 mg/kg group, and Retic (×10¹²/L) increase in the female animals in the 10 mg/kg and 30 mg/kg groups on the next day after the last administration (D30). Peripheral blood CD20⁺ and CD40⁺ B lymphocytes were depleted or decreased, the ratio of CD3⁺ T lymphocyte increased, which are manifestations of pharmacological effects or expanded pharmacological effects of BAT4406F. Anti-drug antibodies were detected in individual animals in each dose group.

Some male animals (⅖) in BAT4406F 30 mg/kg group showed moderate glomerular mesangial hyperplasia, and mild renal tubular dilatation. Electron microscopy examination and immunohistochemical staining results both showed deposition of immune complexes in the kidney glomeruli of the kidneys of animals. Some animals in BAT4406F 10 mg/kg, 30 mg/kg and 100 mg/kg group ( 1/10, 1/10 and 2/10 respectively) showed visible epidermal necrosis/scarring/thickening, acute inflammation of the epidermis and dermis, dermis and subcutaneous subacute or chronic active inflammation of the forefoot or hindfoot skin, all accompanied by vasculitis and/or perivascular inflammation, which were considered to be secondary infections caused by skin trauma and pharmacological effects. At the end of the 20-week recovery period (D169), other lesions had recovered, except that the spleen of some animals ( 2/10) of the BAT4406F 100 mg/kg group still showed drug-related lesions.

The NOAEL (no observed adverse effect level) of this test was considered to be 10 mg/kg.

4. Pharmacokinetic Study

The exposure of BAT4406F in cynomolgus monkeys increased with dose increase, and no significant gender differences in the main pharmacokinetic parameters (Cₘₐₓ, AUC, MRT) were observed in each dose group, after BAT4406F was administered by intravenous infusion to cynomolgus monkeys at a dose of 2 mg/kg, 5 mg/kg, 15 mg/kg or 10-100 mg/kg once a week for 4 consecutive weeks. Since antibodies were produced in the animals two weeks after BAT4406F administration, the drug concentration at the end of the concentration-time curve decreased significantly, so the elimination, half-life, clearance and other parameters are for reference only. See Table 19 and Table 20 for details.

TABLE 19
Pharmacokinetic parameters of BAT4406F following a single intravenous infusion in cynomolgus monkeys

| | | | | t_{1/2} | Tₘₐₓ | Cₘₐₓ | AUC_{(0-672 h)} | AUC_{INF} | Vz | CLₜ | MRT |
|---|---|---|---|---|---|---|---|---|---|---|---|
| Dosage | | Gender | | h | h | μg/mL | h*mg/mL | h*mg/mL | mL/kg | mL/h/kg | H |
| 2 | mg/kg | ♂ | Mean | 66.29 | 1.56 | 48.31 | 2.79 | 2.90 | 65.60 | 0.70 | 77.70 |
| | | | SD | 18.41 | 0.77 | 7.72 | 0.39 | 0.39 | 14.50 | 0.10 | 14.84 |
| | | ♀ | Mean | 59.17 | 0.67 | 49.55 | 2.89 | 2.98 | 56.52 | 0.68 | 82.77 |
| | | | SD | 20.81 | 0.00 | 8.85 | 0.24 | 0.29 | 15.97 | 0.07 | 11.35 |
| 5 | mg/kg | ♂ | Mean | 91.35 | 1.11 | 114.82 | 10.64 | 11.36 | 59.58 | 0.45 | 106.22 |
| | | | SD | 2.33 | 0.77 | 4.59 | 2.09 | 2.27 | 11.85 | 0.10 | 5.60 |
| | | ♀ | Mean | 94.12 | 0.67 | 104.12 | 11.37 | 11.60 | 58.42 | 0.44 | 141.59 |
| | | | SD | 24.57 | 0.00 | 2.98 | 2.27 | 2.17 | 8.78 | 0.08 | 33.66 |
| 15 | mg/kg | ♂ | Mean | 145.62 | 2.00 | 427.95 | 61.21 | 64.19 | 49.18 | 0.24 | 183.98 |
| | | | SD | 58.45 | 0.00 | 56.01 | 5.82 | 6.80 | 18.99 | 0.03 | 16.59 |
| | | ♀ | Mean | 125.49 | 0.67 | 435.93 | 55.74 | 57.85 | 46.13 | 0.26 | 163.77 |
| | | | SD | 44.35 | 0.00 | 44.36 | 6.77 | 8.79 | 9.89 | 0.04 | 26.92 |

TABLE 20
Pharmacokinetic parameters of BAT4406F following multiple
intravenous infusions in cynomolgus monkeys

| | | | | t_{1/2} | Tₘₐₓ | Cₘₐₓ | AUC_{0-168 h} | AUC_{INF} | |
|---|---|---|---|---|---|---|---|---|---|
| Group | | Time | Gender | h | h | μg/mL | h*μg/mL | h*μg/mL | AI |
| 10 | mg/kg | D 1 | ♂ | 146.42 | 0.93 | 233.78 | 16261.70 | 29239.80 | |
| | | | ♀ | 103.29 | 14.93 | 186.76 | 15905.67 | 23954.12 | |
| | | | | 103.29* | 0.67* | 207.34* | 16168.65* | 23954.12* | |
| | | D 22 | ♂ | 187.34 | 5.07 | 433.17 | 41046.27 | 86068.40 | 2.52 |
| | | | ♀ | 153.01 | 8.53 | 446.41 | 39996.58 | 75210.77 | 2.51 |
| | | | | | | | | | 2.47* |
| 30 | mg/kg | D 1 | ♂ | 152.51 | 0.67 | 778.64 | 53259.47 | 96045.93 | |
| | | | ♀ | 128.88 | 0.93 | 721.85 | 50205.85 | 85450.87 | |
| | | D 22 | ♂ | 90.64 | 0.93 | 1026.67 | 96690.21 | 147036.49 | 1.82 |
| | | | ♀ | 113.97 | 0.93 | 890.33 | 70466.82 | 119147.35 | 1.40 |
| 100 | mg/kg | D 1 | ♂ | 141.44 | 0.93 | 2020.91 | 161261.91 | 278066.13 | |
| | | | ♀ | 160.97 | 1.47 | 2072.80 | 151576.43 | 292275.56 | |
| | | D 22 | ♂ | 368.74 | 0.67 | 4271.61 | 371268.10 | 1273968.89 | 2.30 |
| | | | ♀ | 259.40 | 4.40 | 5504.20 | 365303.29 | 1087158.88 | 2.41 |

*The blood concentration of one animal in the first low dose group was abnormal compared with other animals in the same group. It was concluded from the data that some drugs could be infused subcutaneously during the administration and slowly absorbed into the blood, resulting in a significant absorption process before 8 h. Thus, the data was excluded. AI is the ratio of AUC_{0-168 h} after the fourth drug administration and AUC_{0-168 h} after the first drug administration.

### Appendix 1: International Consensus on the Diagnostic Criteria for Neuromyelitis Optica Spectrum Disorders in 2015 (IPND, 2015)

Diagnostic Criteria for NMOSD with AQP4-IgG
(1) At least 1 core clinical characteristic
(2) Positive test for AQP4-IgG using best available detection method (cell-based assay strongly recommended)
(3) Exclusion of alternative diagnoses

Diagnostic Criteria for NMOSD Without AQP4-IgG or Unknown AQP4-IgG Status
(1) At least 2 core clinical characteristics occurring as a result of one or more clinical attacks and meeting all of the following requirements: 1) At least 1 core clinical characteristic must be optic neuritis, acute myelitis with LETM, or area postrema syndrome; 2) Dissemination in space (2 or more core clinical characteristics); 3) Fulfillment of additional MRI requirements
(2) Negative test for AQP4-IgG using best available detection method, or testing unavailable
(3) Exclusion of alternative diagnoses

Core Clinical Characteristics
(1) Optic neuritis
(2) Acute myelitis
(3) Area postrema syndrome: episode of otherwise unexplained hiccups, nausea, vomiting
(4) Acute brainstem syndrome
(5) Symptomatic narcolepsy, diencephalic syndrome, brain MRI with NMOSD-typical diencephalic lesions
(6) Cerebral syndrome with NMOSD-typical brain lesions

Additional MRI Requirements for NMOSD without AQP4-IgG or Unknown AQP4-IgG Status
(1) Acute optic neuritis: requires brain MRI showing one of the following: 1) normal brain MRI or only nonspecific white matter lesions; 2) optic nerve with T2-hyperintense lesion or T1-enhancing lesion extending over ½ optic nerve length or involving optic chiasm
(2) Acute myelitis: requires associated intramedullary lesion extending over 3 contiguous vertebral segments OR 3 or more contiguous vertebral segments of focal spinal cord atrophy in patients with myelitis history
(3) Area postrema syndrome: requires associated dorsal medulla/area postrema lesions
(4) Acute brainstem syndrome: requires associated periependymal brainstem lesions

### Appendix 2: Expanded Disability Scale Score (EDSS)

0 Normal neurological exam (all FS grade 0)
1.0 No disability, minimal signs in one FS (one FS grade 1)
1.5 No disability, minimal signs in more than one FS (more than one FS grade 1)
2.0 Minimal disability in one FS (one FS grade 2, others 0 or 1)
2.5 Minimal disability in two FS (two FS grade 2, others 0 or 1)
3.0 Moderate disability in one FS (one FS grade 3, others 0 or 1) though fully ambulatory; or mild disability in three or four FS (three/four FS grade 2, others 0 or 1) though fully ambulatory
3.5 Fully ambulatory but with moderate disability in one FS (one FS grade 3) and mild disability in one or two FS (one/two FS grade 2) and others 0 or 1; or fully ambulatory with two FS grade 3 (others 0 or 1); or fully ambulatory with five FS grade 2 (others 0 or 1)
4.0 Ambulatory without aid or rest for ≥500 meters; up and about some 12 hours a day despite relatively severe disability consisting of one FS grade 4 (others 0 or 1) or combination of lesser grades exceeding limits of previous steps
4.5 Ambulatory without aid or rest for 300 meters; up and about some 12 hours a day despite relatively severe disability consisting of one FS grade 4 (others 0 or 1) or combination of lesser grades exceeding limits of previous steps
5.0 Ambulatory without aid or rest for ≥200 meters (usual FS equivalents include at least one FS grade 5, or combination of lesser grades usually exceeding specification for step 4.5)
5.5 Ambulatory without aid or rest for ≥100 meters
6.0 Unilateral assistance (cane or crutch) requires to walk at least 100 meters with or without resting
6.5 Constant bilateral assistance (cane or crutch) requires to walk at least 20 meters with or without resting
7.0 Unable to walk beyond 5 meters even with aid. Essentially restricted to wheelchair; wheels self and transfers alone; up and about in wheelchair some 12 hours a day
7.5 Unable to take more than a few steps. Restricted to wheelchair and may need some help in transferring and in wheeling self
8.0 Essentially restricted to bed or chair or perambulated in wheelchair, but out of bed most of the day. Retains many self-care functions. Generally has effective use of arms
8.5 Essentially restricted to bed much of day; Has some effective use of arm(s); retains some self-care functions
9.0 Helpless bed patient. Can still communicate and eat
9.5 Totally helpless bed patient. Unable to communicate effectively or eat/swallow
10 Death due to multiple sclerosis

What is claimed is:

1. A liquid formulation comprising 15 mg/ml to 80 mg/ml of an anti-CD20 antibody, 10 mM to 30 mM buffer, 80 mM to 240 mM stabilizer and 0.1 mg/ml to 0.4 mg/ml surfactant, wherein the pH of the liquid formulation ranges from 5.5 to 6.2;
the buffer is selected from succinate buffer, citrate buffer, phosphate buffer, histidine buffer and acetate buffer;
the stabilizer is selected from sucrose, trehalose, sorbitol, mannitol and methionine; and
the surfactant is selected from polysorbate-80 and polysorbate-20.

2. The liquid formulation according to claim 1, wherein the anti-CD20 antibody is selected from a monoclonal antibody and a CD20-binding fragment.

3. The liquid formulation according to claim 1 or 2, wherein the anti-CD20 antibody comprises:
a light chain variable region (VL) comprising the amino acid sequence of SEQ ID NO: 1 or a polypeptide having at least 80% sequence identity to SEQ ID NO: 1 and a heavy chain variant region (VH) comprising an amino acid sequence of SEQ ID NO: 2 or a polypeptide having at least 80% sequence identity to SEQ ID NO: 2.

4. The liquid formulation according to claim 1 or 2, wherein the anti-CD20 antibody comprises:
a light chain variable region (VL) comprising the amino acid sequence of SEQ ID NO: 1 and a heavy chain variant region (VH) comprising the amino acid sequence of SEQ ID NO: 2.

5. The liquid formulation according to claim 1 or 2, wherein the anti-CD20 antibody comprises:
a light chain comprising the amino acid sequence as shown in SEQ ID NO: 3 or an amino acid sequence having at least 90% sequence identity to SEQ ID NO: 3 and a heavy chain comprising the amino acid sequence as shown in SEQ ID NO: 4 or an amino acid sequence having at least 90% sequence identity to SEQ ID NO: 4.

6. The liquid formulation according to claim 1 or 2, wherein the anti-CD20 antibody comprises:
a light chain comprising the amino acid sequence of SEQ ID NO: 3 and a heavy chain comprising the amino acid sequence of SEQ ID NO: 4.

7. The liquid formulation according to any one of claims 1 to 6, wherein at least about 60% of the anti-CD20 antibody has the G0 glycan at the N-glycosylation site of the constant region of the anti-CD20 antibody.

8. The liquid formulation according to any one of claims 1 to 7, wherein the fucose content of the anti-CD20 antibody is no more than about 5%.

9. The liquid formulation according to any one of claims 1 to 8, wherein the anti-CD20 antibody is produced from a CHO cell line having α-(1,6)-fucosyltransferase gene knocked out.

10. The liquid formulation according to any one of claims 1 to 9, wherein the liquid formulation comprises 20 mg/ml to 50 mg/ml anti-CD20 antibody.

11. The liquid formulation according to any one of claims 1 to 10, wherein the liquid formulation comprises 18 mM to 22 mM histidine buffer.

12. The liquid formulation according to any one of claims 1 to 11, wherein the liquid formulation comprises 158 mM to 225 mM trehalose.

13. The liquid formulation according to any one of claims 1 to 12, wherein the liquid formulation comprises 0.18 mg/ml to 0.22 mg/ml polysorbate-80.

14. The liquid formulation according to any one of claims 1 to 13, wherein the pH of the liquid formulation ranges from 5.7 to 5.9.

15. The liquid formulation according to claim 1, comprising 20 mg/ml anti-CD20 antibody, 20 mM histidine buffer, 158.6 mM trehalose and 0.2 mg/nil polysorbate-80, with the pH of the liquid formulation being 5.8.

16. The liquid formulation according to claim 1, comprising: 20 mg/nil anti-CD20 antibody, 20 mM histidine buffer, 224.6 mM trehalose and 0.2 mg/nil polysorbate-80, with the pH of the liquid formulation being 5.8.

17. The liquid formulation according to claim 1, comprising: 50 mg/ml anti-CD20 antibody, 20 mM histidine buffer, 224.6 mM trehalose and 0.2 mg/nil polysorbate-80, with the pH of the liquid formulation being 5.8.

18. A method for treating a disease involving cells expressing CD20 in a patient in need thereof, comprising administering to the patient an effective amount of the liquid formulation according to any one of claims 1 to 17.

19. The method according to claim 18, wherein the formulation is administrated by intravenous or subcutaneous injection.

20. The method according to claim 18 or 19, wherein the disease involving cells expressing CD20 is selected from a tumorigenic disease and an immune disease.

21. The method according to claim 20, wherein the tumorigenic disease comprises B-cell lymphoma, and the B-cell lymphoma comprises Hodgkin's lymphoma and non-Hodgkin's lymphoma; and the immune disease comprises multiple sclerosis, immune thrombocytopenia, neuromyelitis optica, myasthenia gravis, rheumatoid arthritis, psoriasis and psoriatic arthritis.

22. The method according to claim 18, wherein the disease is neuromyelitis optica spectrum disorders (NMOSD), non-Hodgkin's lymphoma (NHL), multiple sclerosis (MS), immune thrombocytopenia (ITP), rheumatoid arthritis (RA), Wegener's granulomatosis (WG), microscopic polyangiitis (MPA), lupus nephritis, systemic lupus erythematosus or chronic lymphocytic leukemia (CLL).

23. The method according to any one of claims 18 to 22, wherein the disease is refractory to or relapsed after at least one prior therapeutic regimen.

24. Use of the liquid formulation according to any one of claims 1 to 17 for the preparation of a medicament for treating a disease involving cells expressing CD20 in a patient, wherein the disease involving cells expressing CD20 in a patient is selected from a tumorigenic disease and an immune disease.

25. The use according to claim 24, wherein the tumorigenic disease comprises B-cell lymphoma, and the B-cell lymphoma comprises Hodgkin's lymphoma and non-Hodgkin's lymphoma; and the immune disease comprises multiple sclerosis, immune thrombocytopenia, neuromyelitis optica, myasthenia gravis, rheumatoid arthritis, psoriasis and psoriatic arthritis.

26. A method for treating a disease comprising: administering to a patient in need thereof an effective amount of an anti-CD20 mAb or an antigen-binding fragment thereof, which comprises:
a light chain variable region (VL) comprising the amino acid sequence of SEQ ID NO: 1 or a polypeptide having at least 80% sequence identity to SEQ ID NO: 1; and
a heavy chain variant region (VH) comprising an amino acid sequence of SEQ ID NO: 2 or a polypeptide having at least 80% sequence identity to SEQ ID NO: 2; and
wherein the effective amount is about 10 mg to about 3000 mg per dose.

27. The method according to claim 26, wherein the patient has a disease characterized by CD20 positive cells.

28. The method according to claim 26, wherein the disease is neuromyelitis optica spectrum disorders (NMOSD), non-Hodgkin's lymphoma (NHL), multiple sclerosis (MS), immune thrombocytopenia (ITP), rheumatoid arthritis (RA), Wegener's granulomatosis (WG), microscopic polyangiitis (MPA), lupus nephritis, systemic lupus erythematosus or chronic lymphocytic leukemia (CLL).

29. The method according to claim 26, wherein the disease is neuromyelitis optica spectrum disorders.

30. The method according to claim 26, wherein the effective amount is about 300 mg to about 3000 mg per dose.

31. The method according to any one of claims 26 to 30, wherein the disease is refractory to or relapsed after at least one prior therapeutic regimen.

32. The method according to any one of claims 26 to 30, wherein the VL comprises the amino acid sequence of SEQ ID NO: 1, and wherein the VH comprises the amino acid sequence of SEQ ID NO:2.

33. The method according to any one of claims 26 to 31, wherein the anti-CD20 mAb comprises a light chain comprising the amino acid sequence of SEQ ID NO: 3 and a heavy chain comprising the amino acid sequence of SEQ ID NO: 4.

34. The method according to any one of claims 26 to 33, wherein at least about 60% of the anti-CD20 mAb has the G0 glycan at the N-glycosylation site of the constant region of the anti-CD20 mAb.

35. The method according to any one of claims 26 to 34, wherein the fucose content of the anti-CD20 mAb is no more than about 5%.

36. The method according to any one of claims 26 to 35, wherein the anti-CD20 mAb or the antigen-binding fragment thereof is produced from a CHO cell line having α-(1,6)-fucosyltransferase gene knocked out.

37. The method according to any one of claims 26 to 36, wherein the effective amount is about 100 mg to about 1000 mg once every month, every two months, every three months, every four months, every five months, or every six months.

38. The method according to any one of claims 26 to 36, wherein the effective amount is about 100 mg to about 500 mg once every two months, or every three months.

39. The method according to any one of claims 26 to 36, wherein the effective amount is about 300 mg to about 2000 mg per treatment cycle, wherein a treatment cycle is administered once every month, every two months, every three months, every four months, every five months, every six months, every seven months, every eight months, every nine months, every ten months, every eleven months, or every twelve months.

40. The method according to any one of claims 26 to 36, wherein the effective amount is about 500 mg to about 2000 mg administered as needed due to disease reoccurrence.

41. The method according to any one of claims 26 to 36, wherein the administration is by intravenous injection.

42. The method according to any one of claims 26 to 36, wherein the administration is by subcutaneous injection.

43. A kit, comprising the anti-CD20 mAb or the antigen-binding fragment thereof in an amount of 10 mg to about 3000 mg per dose,
wherein the anti-CD20 mAb or the antigen-binding fragment thereof comprises:
a light chain variable region (VL) comprising the amino acid sequence of SEQ ID NO: 1 or a polypeptide having at least 80% sequence identity to SEQ ID NO: 1; and
a heavy chain variant region (VH) comprising an amino acid sequence of SEQ ID NO: 2 or a polypeptide having at least 80% sequence identity to SEQ ID NO: 2.

44. Use of the anti-CD20 mAb or the antigen-binding fragment thereof in an amount of 10 mg to about 3000 mg per dose in the manufacture of a medicament in the treatment of a disease,
wherein the anti-CD20 mAb or the antigen-binding fragment thereof comprises:
a light chain variable region (VL) comprising the amino acid sequence of SEQ ID NO: 1 or a polypeptide having at least 80% sequence identity to SEQ ID NO: 1; and
a heavy chain variant region (VH) comprising an amino acid sequence of SEQ ID NO: 2 or a polypeptide having at least 80% sequence identity to SEQ ID NO: 2.

45. The use according to claim 44, wherein the disease is characterized by CD20 positive cells.

46. The use according to claim 45, wherein the disease is neuromyelitis optica spectrum disorders (NMOSD), non-Hodgkin's lymphoma (NHL), multiple sclerosis (MS), immune thrombocytopenia (ITP), rheumatoid arthritis (RA), Wegener's granulomatosis (WG), microscopic polyangiitis (MPA), lupus nephritis, systemic lupus erythematosus or chronic lymphocytic leukemia (CLL).

47. A pharmaceutical composition, comprising the anti-CD20 mAb or the antigen-binding fragment thereof in an amount of 10 mg to about 3000 mg per dose and a pharmaceutically acceptable carrier,
wherein the anti-CD20 mAb or the antigen-binding fragment thereof comprises:
a light chain variable region (VL) comprising the amino acid sequence of SEQ ID NO: 1 or a polypeptide having at least 80% sequence identity to SEQ ID NO: 1; and
a heavy chain variant region (VH) comprising an amino acid sequence of SEQ ID NO: 2 or a polypeptide having at least 80% sequence identity to SEQ ID NO: 2.

48. A method of producing the pharmaceutical composition according to claim 47, comprising admixing the anti-CD20 mAb or the antigen-binding fragment thereof and the pharmaceutically acceptable carrier.
